(19) United States
(12) Patent Application Publication
(10) Pub. No.: US 2024/0241141 A1
(43) Pub. Date: Jul. 18, 2024

(54) SYSTEM AND METHOD FOR OPTICAL DETECTION OF HEMOGLOBIN VARIANTS, OXYGEN AFFINITY, AND DEOXYGENATION

(71) Applicant: CASE WESTERN RESERVE UNIVERSITY, Cleveland, OH (US)

(72) Inventors: Umut Gurkan, Shaker Heights, OH (US); Zoe Sekyonda, Cleveland, OH (US); Ozan Akkus, Cleveland, OH (US); Ran An, Shaker Heights, OH (US)

(21) Appl. No.: 18/563,113

(22) PCT Filed: May 23, 2022
(86) PCT No.: PCT/US2022/030561

§ 371 Date: Nov. 21, 2023

Related U.S. Application Data
(60) Provisional application No. 63/191,469, filed on May 21, 2021.

(51) Int. Cl. G01N 33/72; G01N 33/49

(52) U.S. Cl. CPC G01N 33/721; G01N 33/49; G01N 2800/22

(57) ABSTRACT

A method of determining at least one of hemoglobin oxygen affinity, rate of hemoglobin deoxygenation, or the presence of hemoglobin variants in blood of a subject, the method includes determining differences of absorption spectra of oxygenated and deoxygenated hemoglobin, red blood, and/or blood obtained from the subject and comparing the determined absorption spectra differences to a control value, wherein the absorption spectra differences are indicative of hemoglobin oxygen affinity, rate of hemoglobin deoxygenation, or the presence of hemoglobin variants in the blood of the subject.

### RELATED APPLICATION

This application claims priority from U.S. Provisional Application No. 63/191,469 filed May 21, 2011, the subject matter of which is incorporated herein by reference in its entirety.

### GOVERNMENT FUNDING

This invention was made with government support under HL133574 awarded by The National Institutes of Health. The government has certain rights in the invention.

### TECHNICAL FIELD

This application describes systems and methods for optical detection of hemoglobin variants, oxygen affinity, and deoxygenation, and particularly relates to a hemoglobin variant detection approach, which will allow rapid screening of hemoglobin disorders.

### BACKGROUND

As blood flows through the lungs, pH rises with an increase in hemoglobin (Hb) oxygen affinity permitting oxygen loading onto Hb; while the blood pH drops in tissue capillaries with decreasing Hb oxygen affinity, facilitating oxygen offloading. The Bohr effects describes Hb's low oxygen affinity with decreased pH. A single point mutation that results in the substitution of valine for glutamic acid at the β-globin subunits of normal hemoglobin (HbA) yields sickle hemoglobin (HbS), which has an altered oxygen affinity. HbS has an abnormally increased Bohr effect that induces oxygen desaturation and can lead to HbS polymerization upon deoxygenation. It is this property of HbS that causes sickle cell disease (SCD).

The HbS oxygen affinity is characterized by an in vitro oxygen dissociation curve (ODC) right shift or a single metric measurement of increased P50 value, which is defined as the partial pressure of oxygen (pO₂) at which 50% of the HbS is saturated with oxygen at temperature of 37° C. and pH of 7.40. However, the in vivo HbS oxygen affinity cannot be characterized solely by standardized environmental conditions since various additional parameters, such as decreased intercellular pH, HbS concentration, polymerization, and elevated 2,3-diphosphoglycerate (2-3DPG), impact the HbS oxygen affinity. Although it is unknown how much variance some of these factors induce in HbS affinity, it is well recognized that the influence of those factors relates to increasing HbS polymerization and decreasing blood pH.

### SUMMARY

Embodiments described herein relate to methods and systems of determining at least one of hemoglobin oxygen affinity, rate of hemoglobin deoxygenation, or the presence of hemoglobin variants in blood of a subject. The methods and systems take advantage of differing Hb oxygenation and deoxygenation optical absorption spectra. It was found that under specific deoxygenation and pH levels, the absorption spectra of HbS exhibit right peak wavelength shift (bathochromic shift) and reduction in optical density (hypochromic shifts) that differ from normal hemoglobin (HbA). Absorption spectra of sickle cell disease (SCD) samples had a larger bathochromic and hypochromic shift magnitude under deoxygenation at all pH levels compared to normal healthy blood containing HbA, where the higher the magnitude of the shift, the lower the oxygen affinity of HbS. The magnitude of bathochromic shift in SCD samples was significantly correlated to the percentage of HbS. HbS bathochromic shift was associated with HbS concentration, suggesting that the low oxygen affinity of HbS, is influenced by the reduced pH and polymerization, which is HbS concentration dependent. In addition, the utility of the method described herein was assessed by the changes in the rate of deoxygenation with oxygen hemoglobin modifying drugs. By direct measurement of oxygen affinity, the methods and systems described herein can add a dimension to measure HbS polymerization, which has clinical implications for evaluating emerging hemoglobin modifying therapies for sickle cell disease.

In some embodiments, a method of determining at least one of hemoglobin oxygen affinity, rate of hemoglobin deoxygenation, or the presence of hemoglobin variants in blood of a subject can include determining differences of absorption spectra of oxygenated and deoxygenated hemoglobin, red blood, and/or blood obtained from the subject. The determined absorption spectra differences can be compared to a control value, wherein the absorption spectra differences are indicative of hemoglobin oxygen affinity, rate of hemoglobin deoxygenation, or the presence of hemoglobin variants in the blood of the subject.

The absorption spectra of the oxygenated and deoxygenated hemoglobin can be measured at the same pH, for example, from about 6.5 to about 9.0, preferably about 6.8 to less than 7.35 or greater than about 7.45 to less than 8.5, or more preferably, about 6.86 or 8.0.

In some embodiments, the differences of absorption spectra are determined by generating a first optical absorption spectra of oxygenated hemoglobin, red blood, and/or blood obtained from the subject, generating a second optical absorption spectra of deoxygenated hemoglobin, red blood, and/or blood obtained from the subject, and comparing the first optical absorption spectra with the second optical absorption to determine differences of the absorption spectra.

In some embodiments, the differences of the absorption spectra include at least one of a bathochromic shift and/or hypochromic shift in peak wavelength from the first absorption spectra to the second absorption spectra.

In some embodiments, the magnitude of barochromic shift in peak wavelength is indicative of at least one of hemoglobin oxygen affinity, rate of hemoglobin deoxygenation, or presence and/or percentage of hemoglobin variants in the hemoglobin, red blood cells, or blood of the subject.

In some embodiments, an increase in magnitude of biochromatic shifty and/or hypochromic shift in peak wavelength is indicative of decreased hemoglobin oxygen affinity, increased hemoglobin deoxidation, or the subject having sickle cell disease.

In some embodiments, the differences of area under a curve of and/or full width at half maximum of peak wavelengths of the first absorption spectra and the second absorption spectra are indicative of anemia of the subject and homogeneity of hemoglobin in the subject.

In some embodiments, the hemoglobin, red blood cells, and/or blood can be deoxygenated by mixing the hemoglobin, red blood cells, and/or blood with an amount of chemical deoxygenant effective to deplete oxygen from the hemoglobin. The chemical deoxygenant can include, for example, sodium metabisulfite.

In other embodiments, the hemoglobin, red blood cells, and/or blood can be deoxygenated by mixing the hemoglobin, red blood cells, and/or blood with an amount of enzymatic deoxygenant effective to deplete oxygen from the hemoglobin. The enzymatic deoxygenant can include, for example, EC-oxyrase.

In some embodiments, the at least one of a bathochromic shift and/or hypochromic shift in peak wavelength of an absorption spectra of hemoglobin, RBC, and/or blood from the subject can be used to detect the presence or quantity hemoglobin variants in the subject, where each hemoglobin variant has a bathochromic shift and/or hypochromic shift in peak wavelength that is unique to and can be used to detect and quantify the hemoglobin variant. The hemoglobin variant detected or quantified can be selected from HbSA, HbSS, HbSC, and HbA2.

In some embodiments, detection of HbSA hemoglobin variant diagnoses the subject as having a sickle cell trait.

In other embodiments, detection of HbSS hemoglobin variant diagnoses the subject as having a sickle cell disease.

In some embodiments, detection of HbSC hemoglobin variant diagnoses the subject as having a hemoglobin SC disease.

In some embodiments, detection of HbA2 hemoglobin variant diagnoses the subject as having thalassemia.

Other embodiments relate to a method of detecting hemoglobin variants in blood of a subject. The method can include determining an optical signature of hemoglobin, red blood cells, and/or blood obtained from the subject that has been deoxygenated, for example, by chemical or enzymatic deoxygenation, and that has a pH from about 6.5 to about 9.0, preferably about 6.8 to less than 7.35 or greater than about 7.45 to less than 8.5, or more preferably, about 6.86 or 8.0. The determined optical signature can then be compared to a control optical signature wherein differences between the determined optical signature and the control optical signature is indicative of hemoglobin variants.

In some embodiments, the optical signature is determined using UV-VIS light spectroscopy.

In some embodiments, the determined optical signature includes an absorption spectra of the deoxygenated hemoglobin, red blood cells, and/or blood.

In some embodiments, the control optical signature includes an absorption spectra of deoxygenated normal hemoglobin, red blood cells, and/or blood obtained at substantially the same pH as the optical signature of the hemoglobin, red blood cells, and/or blood obtained from the subject.

In some embodiments, the method further includes adding hemoglobin, red blood cells, and/or blood obtained from a subject to a pH buffer solution prior to determining the optical signature, wherein the hemoglobin, red blood cells, and/or blood added to the pH buffer solution undergoes a conformational change. The buffer solution can have a weak acidic or weak basic pH, preferably a pH of 6.86 or a pH of 8.0.

In some embodiments, the hemoglobin, red blood cells, and/or blood can be deoxygenated by mixing the hemoglobin, red blood cells, and/or blood with an amount of chemical deoxygenant effective to deplete oxygen from the hemoglobin. The chemical deoxygenant can include, for example, sodium metabisulfite.

In other embodiments, the hemoglobin, red blood cells, and/or blood can be deoxygenated by mixing the hemoglobin, red blood cells, and/or blood with an amount of enzymatic deoxygenant effective to deplete oxygen from the hemoglobin. The enzymatic deoxygenant can include, for example, EC-oxyrase.

In some embodiments, the optical signature, e.g., at least one of a bathochromic shift and/or hypochromic shift in peak wavelength of an absorption spectra of hemoglobin, RBC, and/or blood from the subject, can be used to detect the presence or quantity hemoglobin variants in the subject, where each hemoglobin variant has an optical signature that is unique to and can be used to detect and quantify the hemoglobin variant. The hemoglobin variant detected or quantified can be selected from HbSA, HbSS, HbSC, and HbA2.

In some embodiments, detection of HbSA hemoglobin variant diagnoses the subject as having a sickle cell trait.

In other embodiments, detection of HbSS hemoglobin variant diagnoses the subject as having a sickle cell disease.

In some embodiments, detection of HbSC hemoglobin variant diagnoses the subject as having a hemoglobin SC disease.

In some embodiments, detection of HbA2 hemoglobin variant diagnoses the subject as having thalassemia.

Still other embodiments described herein relate to a system for of determining at least one of hemoglobin oxygen affinity, rate of hemoglobin deoxygenation, or the presence of hemoglobin variants in blood of a subject. The system includes a UV-VIS spectrometer that is configured to determine an optical signature of hemoglobin, red blood cells, and/or blood obtained from the subject that has been deoxygenated and a processor for comparing the determined optical signature to a control optical signature wherein differences between the determined optical signature and the control optical signature is indicative of hemoglobin oxygen affinity, rate of hemoglobin deoxygenation, and/or the presence of hemoglobin variants in the blood of the subject.

In some embodiments, the processor is configured to determine differences of absorption spectra of oxygenated and deoxygenated hemoglobin, red blood, and/or blood obtained from the subject and compare the determined absorption spectra differences to a control value. The absorption spectra differences are indicative of hemoglobin oxygen affinity, rate of hemoglobin deoxygenation, and/or the presence of hemoglobin variants in the blood of the subject.

The absorption spectra of the oxygenated and deoxygenated hemoglobin can be measured at the same pH, for example, from about 6.5 to about 9.0, preferably about 6.8 to less than 7.35 or greater than about 7.45 to less than 8.5, or more preferably, about 6.86 or 8.0.

In some embodiments, the processor is configured to determine differences of absorption spectra by comparing a first optical absorption spectrum of oxygenated hemoglobin, red blood cells, and/or blood with a second optical absorption spectrum of deoxygenated hemoglobin, red blood cells, and/or blood.

In some embodiments, the processor is configured to determine differences in at least one of a bathochromic shift and/or hypochromic shift in peak wavelength from the first absorption spectra to the second absorption spectra.

In some embodiments, the magnitude of barochromic shift in peak wavelength is indicative of at least one of hemoglobin oxygen affinity, rate of hemoglobin deoxygenation, or presence and/or percentage of hemoglobin variants in the hemoglobin, red blood cells, and/or blood of the subject.

In some embodiments, an increase in magnitude of biochromatic shift and/or hypochromic shift in peak wavelength is indicative of decreased hemoglobin oxygen affinity, increased hemoglobin deoxidation, and/or the subject having sickle cell disease.

In some embodiments, the processor is configured to determine differences of area under a curve of and/or full width half maximum of peak wavelengths of the first absorption spectra and the second absorption spectra to determine anemia of the subject and homogeneity of hemoglobin in the subject.

### BRIEF DESCRIPTION OF THE DRAWINGS

FIG. 1 illustrates a flow chart showing a method in accordance with an embodiment.

FIG. 2 illustrates a block diagram showing a system in accordance with an embodiment.

FIGS. 3(A-C) illustrate sequential experimental overview for analysis of oxygenated and deoxygenated normal and sickle samples to obtain optical variables. (A) Normal and sickle lysed or RBC or whole blood samples were prepared in different pH buffers and incubated at room temperature for 600 seconds. After reaching a 162-mmHg oxygen partial pressure (pO₂), the assays were performed using spectrophotometry. (B) Deoxygenation of prepared lysate, RBC, or whole blood samples in various pH buffers was accomplished by mixing with varying concentrations of sodium metabisulphite Na₂S₂O₅·pO₂ of the deoxygenated samples was maintained in the 162-100 mmHg range. (C) At a read time of 270 seconds, oxygenated spectral plots for normal and sickle lysed or RBC or whole blood from 350-750 nm wavelength range were obtained. Three district peaks were detected, with the greatest peak at 414 nm and the other two-valley peaks at 540 and 576 nm. Two district peaks were found for deoxygenated spectral plots for normal and sickle lysed or RBC or whole blood from 350-750 nm wavelength range, with the highest peak at 414 nm acquired in the oxygenated spectra undergoing a bathochromic shift depending on the sample under examination, and the other two-valley peaks forming a single peak at 560 nm. The bathochromic shifts were systematically evaluated in normal, and sickle lysed, RBC, or whole blood.

FIGS. 4(A-B) illustrate sodium metabisulphite (Na₂S₂O₅) induces oxygen partial pressure (pO₂) reduction in whole blood in varying pH levels. pO₂ was measured in both normal (A), (n=5) and sickle (B), (n=5) whole blood at pH of 6.86, 7.2, 7.4, 8.0 and 10, with Na₂S₂O₅ concentrations ranging from 0 to 0.09 M. For both normal (A), and sickle (B) whole blood. pO₂ was measured in millimeters of mercury (mmHg). The pO₂ for both normal and sickle whole blood was in the 162-160 mmHg at a concentration of (0) Na₂S₂O₅. At all pH levels, as Na₂S₂O₅ concentration increased from (0-0.092 M), there was a reduction in pO₂ of whole blood for both normal and sickle cells (0-0.059 M), followed by a slight increase from (0.059-0.066 M) and a plateau from (0.079-0.092 M) M. As the pH decreased from 8.0, 7.4, 7.2 and 6.86, a decrease was observed in pO₂ of both normal and sickle whole blood.

FIGS. 5(A-E) illustrate absorption peak wavelength shifts for normal (HbA) and sickle hemoglobin (HbS) are distinct under deoxygenation. HbA and HbS absorption spectral plots were determined from oxygenated HbA and HbS purified hemoglobin (no Na₂S₂O₅) and from deoxygenated HbA and HbS hemoglobin at Na₂S₂O₅=0.053 M, 28 mmHg pO₂, (HbA; n=5 and HbS; n=5). (A) Spectral plot for oxygenated normal (Oxy HbA) and sickle (Oxy HbS) hemoglobin, as well as deoxygenated normal (deoxy HbA) and sickle (deoxy HbS) hemoglobin, in the range 350-750 nm. The 414 nm peak shifted to a different wavelength position for HbA and HbS purified hemoglobin. The two-valley peak of 540 and 576 integrated into one peak at 560 nm which was the same for both HbA and HbS purified hemoglobin. (B) The zoomed-in spectral plot from 380 to 480 nm for both Oxy HbA vs Oxy HbS hemoglobin and deoxy HbA vs deoxy HbS hemoglobin. Both Oxy HbA and Oxy HbS were detected at 414 nm. Deoxy HbA's had a bathochromic shift to 418 nm, while deoxy HbS's had a bathochromic shift to 424 nm. (C) The bathochromic shift was computed using the deoxygenated spectra and the Soret 414 nm peak as the reference peak. The peak intensity was also expressed by the deoxygenated absorption optical density. The Soret peak in the deoxygenated spectra was then baselined, smoothed, and analyzed. From the normalized deoxygenate shifted peak, (D) the area under the peak (380-460 nm) and (E) full width at half maximum (FWHM) were computed.

FIGS. 6(A-J) illustrate normal hemoglobin (HbA) and sickle hemoglobin (HbS) demonstrate distinct absorption peak wavelength shifts at combinations of degree of deoxygenation and pH. HbA and HbS purified hemoglobin bathochromic shifts caused by deoxygenation were determined by introducing Na₂S₂O₅ (0-0.092 M), corresponding to pO₂ reduction in the range of 162-10 mmHg at pH of 6.86 (A), 7.2 (C), 7.4 (E), 8.0 (G), and 10 (I). Both HbA and HbS shared the same peak wavelengths of 414 nm without the impact of Na₂S₂O₅, at pO₂ of 162 mmHg under all pH conditions. Reduction in pO₂ from 160-20 mmHg caused bathochromic shift increments of both HbA and HbS purified hemoglobin, followed by a plateau from 20-10 mmHg pO₂, at all pH values except pH=10. Magnitudes of the differences between HbA and HbS hemoglobin bathochromic shifts increased with reduction in pO₂ molarity from 162-10 mmHg. At 28 mmHg pO₂ significant differences of bathochromic shifts were identified between HbA and HbS hemoglobin at pH=6.86 (B) (p=0.001) and pH=8 (H) (p=0.001). No significant differences of peak wavelength shifts were identified between HbA and HbS hemoglobin at pH=7.2 (D) (p=0.41) and pH=7.4 (F) (p=0.001). Irrespective of the decrease in the pO₂ no bathochromic shift was observed at pH=10 (J) and the significant difference is non comparable. At pH=6.86, both HbA and HbS hemoglobin had larger peak wavelength shifts than at any other pH. HbS hemoglobin displays a broader peak shift compared to HbA hemoglobin at pH=6.86, 7.2, 7.4 and 8.0. p-values were calculated using Mann-Whitney Test and Data is mean±SEM.

FIGS. 7(A-D) illustrate normal, sickle RBC and whole blood demonstrate distinct absorption peak wavelength shifts at combinations of deoxygenation and at a pH of 6.86. (A) Normal and sickle RBC bathochromic shifts caused by deoxygenation were determined by introducing Na₂S₂O₅ (0-0.092 M) corresponding to reduction in PO₂ at pH of 6.86. (C) Normal and sickle whole blood bathochromic shifts caused by deoxygenation were determined by introducing Na₂S₂O₅ (0-0.092 M) corresponding to reduction in pO₂ at pH of 6.86. Without the effects of Na₂S₂O₅, at pO₂ of 162 mmHg both RBC and whole blood from normal and sickle cells had the same peak wavelengths of 414 nm. Decrease in the pO₂ caused an increase in both normal and sickle bathochromic shifts for both RBC and whole blood from 162 to 10 mmHg, followed by a plateau from 20-10 mmHg. At 28 mmHg pO₂ there were significant differences in peak wavelength shifts between normal and sickle RBC (B) (p=0.001) and normal and sickle whole blood (D) (p=0.001). The highest magnitude of bathochromic shifts was found with RBC compared to whole blood for both normal and sickle. In comparison to normal RBC and whole blood, sickle RBC and whole blood have a greater magnitude of bathochromic shift. p-values were calculated using one sided Mann-Whitney Test. Standard errors of means determined from bathochromic shift for normal and sickle RBCs and whole blood (Repeats numbers (n) as indicated in each of the panels).

FIGS. 8(A-H) illustrate absorption variables accurately determine hemoglobin concentration, percentage of sickle hemoglobin and can distinguish between normal and sickle whole blood at 28 mmHg PO₂, 0.053 Na₂S₂O₅. (A) Sickle whole blood bathochromic shift was substantially associated with the percentage of hemoglobin S (HbS %) determined with HPLC for each whole blood sample (PCC=0.875, p=0.000, n=20). (B) Normal and Sickle whole blood peak optical absorption intensity were significantly and positively correlated to hemoglobin concentration (g/dl) measured with CBC for each whole blood sample (PCC=0.89, p=0.000, SS; (n=20), AA; (n=7)). In addition to the measured bathochromic shift and peak optical absorption intensity parameters, we obtain two computed parameters: the area under the shifted peak and the shifted peak full width at half maximum (FWHM)(nm). (C) Area under the shifted peak were significantly and inversely associated to bathochromic shift for both normal and sickle whole blood at 28 mmHg pO₂, (PCC=−0.79, p=0.000, SS(n=20), AA (n=17)). (D) FWHM were significantly but not strongly associated with peak wavelength shift for both normal and sickle whole blood, (PCC=0.22, p=0.009, SS(n=20), AA (n=17)). Comparison of optical parameters in normal and sickle whole blood (E) Sickle whole blood had a higher magnitude of bathochromic shift than normal blood:(sickle 420.6±0.44, normal; 415.3.0±0.21), (F) Normal whole blood had a higher shifted peak optical absorption intensity than sickle whole blood, (sickle; 0.99±0.04, normal 2.0±0.03), (H) Normal whole blood had a higher area under the shifted peak density than sickle blood, (sickle 11.6±0.69, normal 37.2±0.8) and (G) sickle whole blood had a higher shifted peak FWHM than normal blood (sickle 30.9±0.84, normal; 24.3±0.82). Significant difference of all the optical parameters were identified between normal and sickle whole blood (E) peak wavelength shift (p=0.001), (F) shifted peak optical density (p=0.000), (H) Area under the shifted peak (p=0.001), and (G) shifted peak FWHM (p=0.001). Sample size (n) as indicated in each of the panel. Pearson Correlation Coefficient, p-value are calculated from linear regression and using Mann-Whitney Test.

FIGS. 9(A-B) illustrate principal component analysis (PCA) can cluster Normal (AA) and sickle (SS)whole blood samples into different groups based on optical variables. (A) The 201-wavelength of shifted peak optical density derived from 350-750 (UV-Visible range) and (B) four optical parameters (peak wavelength, peak shifted optical density, area under the peak and shifted peak (FWHM)) were converted into two linearly uncorrected principal components for both normal (AA) and sickle (SS) whole blood samples at 28 mmHg pO₂. PC1 and PC2 are the first and second principal components, respectively. The two components are then clustered and visualized in two dimensions using PCA. (A) The two components account for 98.5% variability in the full spectrum peak shifted optical density original data set, with the first component at 77.96% of the total variation between normal (HbA) and sickle (HbS) whole blood samples (B) The two components accounting for 94.5% variability in the optical parameters data set, the first component accounting for 79.38% in the total variation. Only one outliner was found in the full spectrum data set specifically in in normal (HbA) whole blood samples (A), while two were found in the four optical parameters dataset each from normal (HbA) and sickle (HbS) whole blood samples (B). PCA clearly clustered and differentiated normal (AA) and sickle (SS) whole blood samples from any of the generated data set from this study. Sample size (SS=20 and AA=17).

FIGS. 10(A-D) illustrate rate of deoxygenation (Kc) can be determined using the bathochromic and hypochromic shift and voxelotor reduces the Kc of sickle whole blood samples at pH 6.86. (A) Spectral plot of a single whole blood samples from full oxygenation to partial deoxygenation. As the whole blood sample transition from oxygenation to deoxygenation the spectral peak wavelength undergoes a bathochromic shift from 414 nm that was directly propositional to hypochromic shift. Bathochromic and hypochromic shifts were assessed every 30 second for 10 minutes. (B) Using the kinetics of bathochromic and hypochromic shift we determined the rate of deoxygenation plot and by determining the slope of the tangent line, the rate of deoxygenation per second (Kc) can be measured for HbAA, HbAA+ Vox, HbSS and HbSS+ Vox whole blood samples. (C) HbS samples had a significantly increased rate of deoxygenation compared to HbA whole blood samples (p=0.002). However, when HbS were treated with voxelotor to create HbS+ Vox the Kc was significantly reduced by 57.5%±6.7% (p=0.004) but the Kc were still significantly elevated than the Kc for HbA samples (p=0.004). No significate difference was observed been HbAA and HbAA+ Vox samples (p=0.82). (D) The Kc values were found to correspond to the oxygen capacity (g/dl) of the sample. The HbS samples had a higher the Kc but with lower oxygen capacity while HbA samples had the opposite. However, HbS+ Vox had an increased Kc by lower oxygen capacity, thus voxelotor reduced the rate of deoxygenation but did not increase the oxygen capacity. p-value are calculated using Mann-Whitney test and paired t test.

FIG. 11 illustrate mechanism underlying the observed increased bathochromic shifts at pH 6.86 among sickle samples. HbS and HbA had the same bind oxygen at full oxygenation with sample at pO₂ 162 mmHg. Once HbS and HbA were combined in a pH buffer solution of 6.86, the hydrogen ions in the solution increased. The binding of hydrogen ions to the oxygen binding site increased hemoglobin's tendency to release oxygen. When pH 6.86 was compared to other pH values investigated, the behavior of increased oxygen release was seen with increased peak shift wavelength magnitude of hemoglobin. The addition of Na₂S₂O₅ exacerbated the loss of oxygen. Na₂S₂O₅ reduces oxygen to produce peroxides and by replacing oxygen binding sites with sulphate (SO3). Deoxygenating HbS promotes the formation of polymers, which intensifies the amount of oxygen released. The larger peak wavelength shifts observed in sickle samples demonstrated HbS's reduced oxygen affinity, which was correlated to the proportion of HbS in the sample. The lower the affinity of HbS, the greater the peak wavelength shift observed and, the higher the percentage of HbS.

FIG. 12 illustrate pH differences between HbA and HbS whole blood changed significantly with additional of buffer and Na₂S₂O₅. The pH of HbA (n=6) and HbS (n=6) whole blood samples was measured before and after additional to buffer 6.86 and Na₂S₂O₅. The pH of HbA samples at full oxygenation (162 mmHg pO₂) was in the normal range of 7.35 to 7.45 but significantly higher than HbS (p=0.003) that were in the range 7.2-7.14. Additional of 6.86 buffer to the whole blood neutralized the pH of HbS and HbA samples to a baseline pH of 6.86 and no significate differences between HbS and HbA was observed (p=0.65). However additional of 1% Na₂S₂O₅ to the mixture of blood and buffer at 28 mmHg pO₂ reduced the pH of HbS and HbA samples sightly further and the pH of HbS whole blood samples was significantly lower than HbA (p=0.002). p-values were calculated using Mann-Whitney Test.

FIG. 13 illustrate robustness and repeatability of the peak wavelength shift assay. Repeatability test peak wavelength shift was determined from 20 tests using the same samples at 20 mmHg pO₂, comparing variances between 2 users (demonstrated in the FIG. 7). There were 2 samples used (one normal and one sickle). Each sample was assessed 5 times and the same sample was examined in 3 well micro wells throughout each test, thus each user tested 10 times. The peak wavelength shift between the two users demonstrated a strong repeatability (Mean±SEM), (User1: sickle; 421.6±0.24, normal; 414.9±0.11; User2: sickle; 421.1±0.26, normal; 415.1±0.05) and coefficient of variance (COV)=0.05% for SS and 0.02% for AA). There were no significant differences in peak wavelength shifts between User 1 and User 2 for both normal and sickle whole blood (p=0.07) and (p=0.68) respectively. p-values were calculated using Mann-Whitney test.

FIG. 14 illustrates changes in temperature can induce an increase in the magnitude of bathochromic shift in both normal (HbA) and sickle (HbS) whole blood samples. Although most of the measurements in our study were done at room temperatures (25° C.), the influence of body temperature (37° C.) on the extent of bathochromic shift was also investigated. Increase in the magnitude of bathochromic shifts was significate for both HbA and HbS with an increase in temperature from 25 to 37° C., (p=0.001) and (p=0.004) respectively. However, the percentage of bathochromic shifts increment was higher in HbS samples compared to HbA (HbS; 1%±0.12%, HbA; 0.82%±0.18%) increase in temperature influences an increase in the magnitude bathochromic shift. p-values were calculated using paired t test.

FIGS. 15(A-H) illustrate optical absorption shift at 414 nm with chemical deoxygenation accurately identify Hemoglobin Variant. (A) For blood samples containing AA vs SS, shift difference at the 414 nm oxygenation peak with deoxygenation at pH 8.0. The red line depicts how samples containing hemoglobin variant SS have an increased shift as compared to samples containing AA black line. (B) Under pH 6.8, deoxygenation induces a shift difference at the 414 nm oxygenation peak for blood samples containing AA vs SS with a wider shift. (C) Under pH 7.2, While a shift was observed at the 414 nm oxygenation peak with deoxygenation for blood samples containing AA vs SS, a broader shift difference was observed under pH 8 and 6.8. (D) Under pH 10, blood samples containing variant SS shifted indistinguishably from blood samples containing variant AA at pH 10. (E) Compares shift difference for SS, AA and thalassemia under 8.0 and (F) Compares shift difference for SS, AA and thalassemia under pH 6.8. The various variants could be classified and differentiated based on the 414 nm shift, as can be shown. The mean shift difference at chemical deoxygenation of 17 mg under (G) pH 8.0 and (H) pH 6.8 was tested using one-way Mann-Whitney at a 95% confidence interval, p<0.05 indicating a statistical detectable difference between 23 AA samples and 23 SS variant sample.

### DETAILED DESCRIPTION

To facilitate the understanding of this invention, a number of terms are defined below. Terms defined herein have meanings as commonly understood by a person of ordinary skill in the areas relevant to the present invention. Terms such as “a”, “an”, and “the” are not intended to refer to only a singular entity but also plural entities and also includes the general class of which a specific example may be used for illustration. The terminology herein is used to describe specific aspects of the invention, but their usage does not delimit the invention, except as outlined in the claims.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation. “Optional” or “optionally” means that the subsequently described circumstance may or may not occur, so that the description includes instances where the circumstance occurs and instances where it does not.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

Embodiments described herein relate to methods and systems of determining at least one of hemoglobin oxygen affinity, rate of hemoglobin deoxygenation, or the presence of hemoglobin variants in blood of a subject. We found that differing hemoglobin (Hb) oxygenation and deoxygenation optical absorption spectra determined using light absorption spectroscopy can be used to distinguish hemoglobin variants based on their oxygen retention capability. When an optical beam strikes a solution containing deoxygenated hemoglobin, light is absorbed, and the intrinsic hemoglobin molecules interact with light photons energy to produce distinct optical signatures of absorption shifts that are unique to each hemoglobin variant under investigation. We found that under specific deoxygenation and pH levels, the absorption spectra of HbS exhibit right peak wavelength shift (bathochromic shift) and reduction in optical density (hypochromic shifts) that differ from normal hemoglobin (HbA). Absorption spectra of sickle cell disease (SCD) samples had a larger bathochromic and hypochromic shift magnitude under deoxygenation at all pH levels compared to normal healthy blood containing HbA, where the higher the magnitude of the shift, the lower the oxygen affinity of HbS. The magnitude of bathochromic shift in SCD samples was significantly correlated to the percentage of HbS. HbS bathochromic shift was associated to HbS concentration, suggesting that the low oxygen affinity of HbS, is influenced by the reduced pH and polymerization which is HbS concentration dependent. In addition, the method described herein can be used to assess changes in the rate of deoxygenation with oxygen hemoglobin modifying drugs. By direct measurement of oxygen affinity, the methods and systems described herein adds a dimension to measure HbS polymerization, which has clinical implications for evaluating emerging hemoglobin modifying therapies for sickle cell disease.

Accordingly, in some embodiments a method of determining at least one of hemoglobin oxygen affinity, rate of hemoglobin deoxygenation, or the presence of hemoglobin variants in blood of a subject can include determining differences of absorption spectra of oxygenated and deoxygenated hemoglobin, red blood, and/or blood obtained from the subject. The determined absorption spectra differences can be compared to a control value, wherein the absorption spectra differences are indicative of hemoglobin oxygen affinity, rate of hemoglobin deoxygenation, or the presence of hemoglobin variants in the blood of the subject.

The absorption spectra of the oxygenated and deoxygenated hemoglobin can be measured at the same pH, for example, from about 6.5 to about 9.0, preferably about 6.8 to less than 7.35 or greater than about 7.45 to less than 8.5, or more preferably, about 6.86 or 8.0.

In some embodiments, the differences of absorption spectra are determined by generating a first optical absorption spectra of oxygenated hemoglobin, red blood, and/or blood obtained from the subject, generating a second optical absorption spectra of deoxygenated hemoglobin, red blood, and/or blood obtained from the subject, and comparing the first optical absorption spectra with the second optical absorption to determine differences of the absorption spectra.

In some embodiments, the differences of the absorption spectra include at least one of a bathochromic shift and/or hypochromic shift in peak wavelength from the first absorption spectra to the second absorption spectra.

In some embodiments, the magnitude of barochromic shift in peak wavelength is indicative of at least one hemoglobin oxygen affinity, rate of hemoglobin deoxygenation, or presence and/or percentage of hemoglobin variants in the hemoglobin, red blood cells, or blood of the subject.

In some embodiments, an increase in magnitude of biochromatic shifty and/or hypochromic shift in peak wavelength is indicative of decreased hemoglobin oxygen affinity, increased hemoglobin deoxidation, or the subject having sickle cell disease.

In some embodiments, the differences of area under a curve of and/or full width at half maximum of peak wavelengths of the first absorption spectra and the second absorption spectra are indicative of anemia of the subject and homogeneity of hemoglobin in the subject.

In some embodiments, the hemoglobin, red blood cells, and/or blood can be deoxygenated by mixing the hemoglobin, red blood cells, and/or blood with an amount of chemical deoxygenant effective to deplete oxygen from the hemoglobin. The chemical deoxygenant can include, for example, sodium metabisulfite.

In other embodiments, the hemoglobin, red blood cells, and/or blood can be deoxygenated by mixing the hemoglobin, red blood cells, and/or blood with an amount of enzymatic deoxygenant effective to deplete oxygen from the hemoglobin. The enzymatic deoxygenant can include, for example, EC-oxyrase.

In some embodiments, the hemoglobin variant is selected from HbSA, HbSS, HbSC, and HbA2.

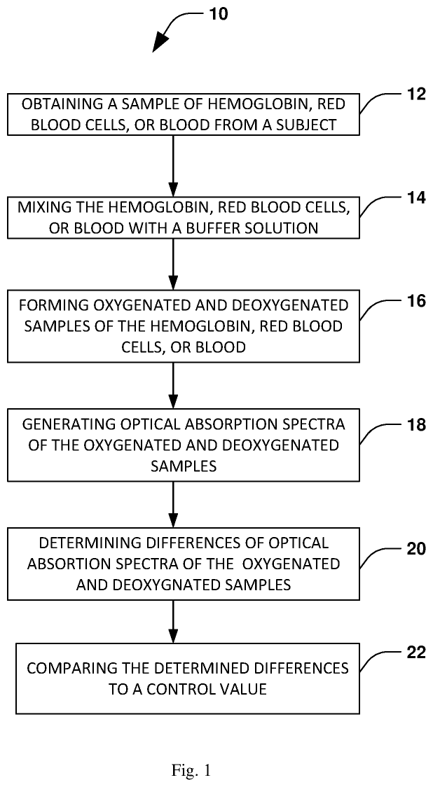

FIG. 1 illustrates a flow chart 10 showing a method of determining at least one of hemoglobin oxygen affinity, rate of hemoglobin deoxygenation, or the presence of hemoglobin variants in blood of a subject.

In the method at step 12, a sample of hemoglobin, red blood cells, or blood can be obtained from a subject. The sample can include whole blood, isolated red blood cells (RBCs), and purified hemoglobin lysed RBCs.

At step 14, the hemoglobin, red blood cells, and/or blood can be mixed with a buffer solution to provide a hemoglobin, red blood cell, or blood suspension with a pH that amplifies the Bohr effect of hemoglobin (Hb) including Hb variants, such as HbS. Any change in pH beyond the physiological range of 7.35 to 7.45 amplifies this Hb Bohr effect.

In some embodiments, the pH of the hemoglobin, red blood cell, or blood suspension can be adjusted with the buffer from a physiological blood pH of about 7.35 to 7.45 to a pH less or greater than physiological pH in a range of about 6.5 to about 9.0. For example, the hemoglobin, red blood cell, or blood suspension can be adjusted with the buffer from physiological blood pH to about 6.8 to less than 7.35 or greater than about 7.45 to less than 8.5, or preferably, about 6.86 or 8.0.

By way of example, whole blood, RBCs, and/or purified Hb can be mixed with pH 6.86 buffer solution and incubated for a duration of time at room temperature. The pH of the buffer suspensions can be checked after addition to the whole blood, RBCs, or Hb.

Following mixing of the Hb, RBCs, or blood with the pH buffer, at step 16, samples of the pH adjusted Hb, RBCs, or blood suspension can be oxygenated and deoxygenated to provide oxygenated and deoxygenated samples of the pH buffered Hb, RBCs, or blood suspension. For example, all samples can initially exposed to ambient air. The oxygen partial pressure (PO₂) which is 21% of atmospheric pressure (773 mmHg), can then normalized to 162 mmHg PO₂ during oxygenation to provide oxygenated samples of the Hb, RBCs, or blood suspension.

Samples of the Hb, RBCs, or blood suspension can be deoxygenated chemically or enzymatically using, for example, sodium metabisulphite (Na₂S₂O₅), sodium dithionite (Na₂S₂O₄), or EC-oxyrase. For example, predetermined amounts of sodium metabisulphite can be mixed with the prepared samples of the Hb, RBCs, or blood suspension to provide deoxygenated samples with a gradual reduction in the oxygen partial pressure and deoxygenation levels.

At step 18, optical absorption spectra of the oxygenated and the deoxygenated Hb, RBC, and/or blood samples can be generated using a UV-VIS. The optical absorption spectra can include a generated first optical absorption spectrum of oxygenated samples of the Hb, RBCs, and/or blood suspension and a generated second optical absorption spectrum of deoxygenated Hb, RBCs, and/or blood suspension. The spectral rang of the absorption spectra can be from about 300 nm to about 800 nm with a resolution of, for example, about 1 nm or 2 nm. For example, during sample testing of either oxygenated and deoxygenated samples, samples can be analyzed in microplate wells using a spectroscopy microplate reader, such as Petromax Me2, (Molecular devices, San Jose, CA), over a spectral range of about 350 nm to about 750 nm, with a wavelength resolution of 2 nm and at a customized microplate well reading setting and at room temperature.

By way of example, fully oxygenated samples were analyzed, to obtain reference signatures or control signature or control optical absorption spectra that included a Soret band (e.g., 380-480 nm) and two peaks in Q-band (e.g., 560-580 nm). All oxygenated samples had their highest absorption oxygenated peak at 414 nm. Following that, the deoxygenated samples were analyzed across the same spectral range as the oxygenated samples, but with Na₂S₂O₅ concentrations varying, for example, from 0.039 to 0.092 M. The concentration of Na₂S₂O₅ used was corresponding to a decrease from 100-0 (mmHg) of pO₂ in samples.

In some embodiments, absorbance measurements are conducted at a substantially constant temperature. As such, the temperature during absorbance measurement changes by 5° C. or less, such as by 4.5° C. or less, such as by 4° C. or less, such as by 3.5° C. or less, such as by 3° C. or less, such as by 2.5° C. or less, such as by 2° C. or less, such as by 1.5° C. or less, such as 1° C. or less, such as by 0.5° C. or less, such as by 0.1° C. or less, such as by 0.05° C. or less, such as by 0.01° C. or less, such as by 0.005° C., such as by 0.001° C., such as by 0.0001° C., such as by 0.00001° C. or less and including by 0.000001° C. or less.

Following generation of the optical absorption spectra of the oxygenated and the deoxygenated samples of Hb, RBCs, and/or blood, at step 20, the optical absorption spectra of the Hb, RBCs, and/or blood can be compared to determine differences in the absorption spectra.

In some embodiments, the differences of the absorption spectra include at least one of a bathochromic shift and/or hypochromic shift in peak wavelength from the first absorption spectra to the second absorption spectra, differences of area under a curve of the first absorption spectra and the second absorption spectra, and/or differences of the full width at half maximum of peak wavelengths of the first absorption spectra and the second absorption spectra.

At step 22, the differences of the absorption spectra can be compared to a control value to determine at least one of hemoglobin oxygen affinity, rate of hemoglobin deoxygenation, or the presence of hemoglobin variants in blood of a subject.

A “control value” or “appropriate standard” is a standard, parameter, value or level indicative of a known outcome, status or result (e.g., a known disease or condition status). A control value or appropriate can be determined (e.g., determined in parallel with a test measurement) or can be pre-existing (e.g., a historical value, etc.). For example, a control value or appropriate standard may be a bathochromic shift and/or hypochromic shift in peak wavelength, differences of area under a curve, and/or differences of the full width at half maximum of peak wavelengths obtained from a subject known to have a sickle cell disease, or a subject identified as being disease-free. In the former case, a lack of a difference between the measured differences in adsorption spectra and the differences in absorption spectra of an appropriate standard may be indicative of a subject having a disease or condition. Whereas in the latter case, the presence of a difference between the measured differences of absorption spectra and the differences of absorption spectra of the control value or appropriate standard may be indicative of a subject having a disease or condition.

The magnitude of a difference between a parameter, level or value the absorption spectra that is indicative of outcome, status or result may vary. For example, a significant difference that indicates a known outcome, status or result may be detected when the level of a parameter, level or value is at least 1%, at least 5%, at least 10%, at least 25%, at least 50%, at least 100%, at least 250%, at least 500%, or at least 1000% higher, or lower, than the appropriate standard. Similarly, a significant difference may be detected when a parameter, level or value is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the level of the appropriate standard. Significant differences may be identified by using an appropriate statistical test. Tests for statistical significance are well known in the art and are exemplified in Applied Statistics for Engineers and Scientists by Petruccelli, Chen and Nandram Reprint Ed. Prentice Hall (1999).

In some embodiments, the magnitude of barochromic shift in peak wavelength compared to a control value or shift is indicative of at least one of hemoglobin oxygen affinity, rate of hemoglobin deoxygenation, or presence and/or percentage of hemoglobin variants in the hemoglobin, red blood cells, and/or blood of the subject. An increase in magnitude of biochromatic shift and/or hypochromic shift in peak wavelength compared to a control value or shift can be indicative of decreased hemoglobin oxygen affinity, increased hemoglobin deoxidation, or the subject having sickle cell disease.

For example, both oxygenated HbS and HbA purified hemoglobin exhibited 3 distinct peaks at 414 nm and two-valley peaks at 540 and 576 nm. The maximum 414 nm peak observed in oxy-HbS and oxy-HbA shifted to different extents upon deoxygenation such that the shift of HbS was greater (424 nm and 418 nm). It was noted that the trends of changes in the wavelength with deoxygenation was similar to the trend of deoxygenation with Na₂S₂O₅ indicating that bathochromic shift is a surrogate marker for deoxygenation. At 162 mm Hg pO₂, both normal and sickle whole blood had a peak wavelength of 414 nm. The reduction in pO₂ from 162 to 10 mmHg in both normal and sickle whole blood caused an increase in shift and quasi-equilibrium was observed at 28 mmHg pO2.

The variations of bathochromic shift shown for purified hemoglobin was also observable for RBCs and whole blood. RBCs and whole blood from normal and SCD patients also exhibited the three wavelengths that were present in purified hemoglobin. From 162 to 10 mmHg pO₂, decrease in PO₂ induced a rise in both normal and sickle samples peak wavelength for both RBCs and whole blood, followed by a plateau from 20−10 mmHg. At 28 mmHg pO₂, bathochromic shifts between normal and sickle RBCs (p=0.001) and normal and sickle whole blood (p=0.001) were significantly different. Compared to bathochromic shift of purified hemoglobin for both HbA and HbS at pH 6.86, RBCs had the largest magnitude of bathochromic shift, followed by purified hemoglobin and then whole blood samples. Sickle RBCs and whole blood had a larger bathochromic shift compared to normal RBCs and whole blood.

In some embodiments, the differences of area under a curve of and/or full width at half maximum of peak wavelengths of the first absorption spectra and the second absorption spectra are indicative of anemia of the subject and homogeneity of hemoglobin in the subject.

In some embodiments, the at least one of a bathochromic shift and/or hypochromic shift in peak wavelength of an absorption spectra of hemoglobin, RBC, and/or blood from the subject can be used to detect the presence or quantity hemoglobin variants in the subject, where each hemoglobin variant has a bathochromic shift and/or hypochromic shift in peak wavelength that is unique to and can be used to detect and quantify the hemoglobin variant. The hemoglobin variant detected or quantified can be selected from HbSA, HbSS, HbSC, and HbA2.

In some embodiments, detection of HbSA hemoglobin variant diagnoses the subject as having a sickle cell trait.

In other embodiments, detection of HbSS hemoglobin variant diagnoses the subject as having a sickle cell disease.

In some embodiments, detection of HbSC hemoglobin variant diagnoses the subject as having a hemoglobin SC disease.

In some embodiments, detection of HbA2 hemoglobin variant diagnoses the subject as having thalassemia.

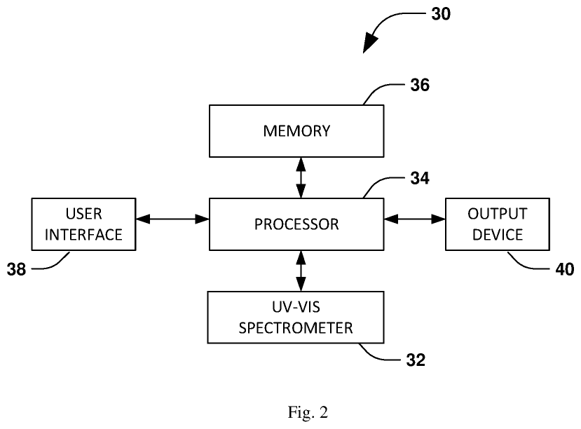

Still other embodiments described herein relate to a system for determining at least one of hemoglobin oxygen affinity, rate of hemoglobin deoxygenation, or the presence of hemoglobin variants in blood of a subject. FIG. 2 illustrates a block diagram of an example of a system 30 for determining at least one of hemoglobin oxygen affinity, rate of hemoglobin deoxygenation, or the presence of hemoglobin variants in blood of a subject. The system 30 includes a UV-VIS spectrometer 32 that is configured to determine an optical signature of hemoglobin, red blood cells, or blood obtained from the subject that has been deoxygenated and a processor or computer processor 34 for comparing the determined optical signature to a control optical signature wherein differences between the determined optical signature and the control optical signature is indicative of hemoglobin oxygen affinity, rate of hemoglobin deoxygenation, or the presence of hemoglobin variants in the blood of the subject.

In some embodiments, the processor 34 is configured to determine differences of absorption spectra of oxygenated and chemically deoxygenated hemoglobin, red blood, and/or blood obtained from the subject and compare the determined absorption spectra differences to a control value. The absorption spectra differences are indicative of hemoglobin oxygen affinity, rate of hemoglobin deoxygenation, or the presence of hemoglobin variants in the blood of the subject.

The processor 34 typically receives and processes optical measurements that are performed by the UV-VIS spectrometer 32. Further typically, the processor 34 controls the acquisition of optical measurements that are performed by the UV-VIS spectrometer. The processor 34 communicates with a memory 36. A user (e.g., a laboratory technician) sends instructions to the computer processor via a user interface 38. For some applications, the user interface includes a keyboard, a mouse, a joystick, a touchscreen device (such as a smartphone or a tablet computer), a touchpad, a trackball, a voice-command interface, and/or other types of user interfaces that are known in the art. Typically, the computer processor generates an output via an output device 40. Further typically, the output device includes a display, such as a monitor, and the output includes an output that is displayed on the display. For some applications, the processor generates an output on a different type of visual, text, graphics, tactile, audio, and/or video output device, e.g., speakers, headphones, a smartphone, or a tablet computer. For some applications, user interface 36 acts as both an input interface and an output interface, i.e., it acts as an input/output interface. For some applications, the processor generates an output on a computer-readable medium (e.g., a non-transitory computer-readable medium), such as a disk, or a portable USB drive, and/or generates an output on a printer.

In some embodiments, the temperature of the system may be controlled by a temperature control subsystem (not shown), which measures the system temperature and if necessary, controls the ambient conditions to maintain a desired system temperature. Temperature subsystems may include any convenient temperature control protocol, including, but not limited to heat sinks, fans, exhaust pumps, vents, refrigeration, coolants, heat exchanges, Peltier or resistive heating elements, among other types of temperature control protocols.

In some embodiments, the UV-VIS spectrometer can include broadband light source (such as a Tungsten Halogen bulb), monochromator that selects certain wavelengths from that broadband light source, a computer than can command the monochromator to select certain wavelengths, a collimating stage that accepts a sample and a detector that characterizes light intensity after it has passed through the sample cell.

In some embodiments, the memory has instructions stored thereon, which when executed by the processor, cause the system to irradiate the sample with the desired wavelengths of light, determine a measured intensity of light at the desired wavelengths, and calculate the absorbance until a spectrum of absorbance vs wavelength covering the desired wavelength region.

In some embodiments, the processor is configured to determine bathochromic shift and/or hypochromic shift in peak wavelength, differences of area under a curve, and/or differences of the full width at half maximum of peak wavelengths.

In some embodiments, a computer readable storage medium may be employed on one or more components of the system having a display and operator input device. Also provided are non-transitory computer readable storage media. Such media can be, for example, a CD-ROM, a USB drive, a floppy disk, or a hard drive. In some cases, the medium comprises instructions stored thereon for separating an absorption spectrum into a Rayleigh scattering contribution and an absorption contribution. In some cases, the instructions comprise: an algorithm for measuring an absorption spectrum (ii) an algorithm for generating a fit spectrum by fitting the absorption spectrum to a power function (iii) an algorithm for generating a difference spectrum by subtracting the fit spectrum from the absorption spectrum (iv) an algorithm for generating an adjusted spectrum by selecting points from the absorption spectrum for wavelengths wherein the difference spectrum is less than or equal to zero points from the fit spectrum for wavelengths wherein the difference spectrum is greater than zero (v) an algorithm for repeating steps (ii)-(iv) zero or more times, wherein the most recent adjusted spectrum is used in place of the absorption spectrum if the steps are repeated.

In some embodiments, the system described herein can employ supervised machine learning. In some embodiments, supervised machine learning can detect difference in the generated or measured absorption spectra. For example, supervised machine learning can detect changes or differences in differences of a bathochromic shift and/or hypochromic shift in peak wavelength from generated or measured absorption spectra. In some embodiments, supervised machine learning can detect changes such as differences of area under a curve of and/or full width at half maximum of peak wavelengths of absorption spectra. In some cases, supervised machine learning can be used to classify samples Hb variant or concentration as well as hemoglobin oxygen affinity or rate of hemoglobin deoxygenation.

### Example 1

This Example describes a new rapid optical diagnostic approach and, rate of deoxygenation and hemoglobin oxygen affinity measurement method. We exploited the differing oxygenation and deoxygenation spectra of Hb to determine the difference in the oxygen affinity and Bohr effect between HbA and HbS in purified hemoglobin, RBCs and in whole blood. Optical absorption spectra of HbA are affected by the oxygenation-deoxygenation dynamics and to any alteration in the Hb confirmation structure. HbA's oxygenated (Oxy-HbA) and deoxygenated (Deoxy-HbA) optical spectra have been vital in measurement of physiological parameters including hemoglobin concentration, noninvasive blood oxygen saturation levels and pulse oximetry. The absorption spectra of deoxygenated and oxygenated hemoglobin can be utilized to monitor hemoglobin's rate of deoxygenation and oxygen affinity. In the presence of certain hemoglobinopathies, the absorption spectra of HbA were found to shift in peak wavelength.

We show that while it has been assumed that the absorption spectral of HbA and HbS are identical, under specific deoxygenation levels and pH, the absorption spectra of HbA and HbS are distinct. HbS exhibited spectra peak wavelength shift to the right (bathochromic shift) and reduction optical density (hypochromic shift) in purified hemoglobin, RBCs, and whole blood that differ from HbA. Leveraging this newly discovered property, initially we used this method to detect HbS. We then applied the bathochromic shifts to evaluate oxygen affinity from whole blood. We found that the magnitude of bathochromic shift was a concomitate to the oxygen affinity. With HbS the spectral bathochromic shift was associated to HbS concentration, suggesting that the low oxygen affinity of HbS, is influenced by the reduced pH and polymerization which is an HbS concentration dependent mechanism. We demonstrated the utility of using our method to measure the rate deoxygenation by describing the kinetics of deoxygenation in whole blood sample with hemoglobin oxygen modifying treatment. We provide an optical approach for in vitro and in vivo diagnostic approaches for sickle hemoglobin, and oxygen affinity and rate of deoxygenation measurement and demonstrate its clinical significance by evaluating the hemoglobin modifying therapies for sickle cell disease both by direct inhibition of polymerization and by increasing the oxygen affinity.

### Materials and Methods

### Materials

Buffer solutions of pH 6.86, 7.2, 7.4, 8.0 and 10.0 were purchased from Fisher Scientific (Pittsburgh, PA). All buffer solutions were stored at room temperature. Sodium metabisulfite (Na₂S₂O₅) was purchased from Sigma Aldrich (St Louis, Mo) to be used for chemical deoxygenation. Nunc polystyrene 90 microwell plate non treated surface with flat bottom and lid were purchased from Thermo Fisher Scientific (Waltham, MA).

### Samples

Blood samples were collected from de-identified healthy donors and SCD patients as part of standard clinical care. All participants in the study provided written informed consent. When blood samples were collected, they were kept at 4 degrees Celsius and processed within 6 hours. Samples were collected in EDTA-containing vacutainer tubes and separated into two groups: Normal samples from healthy donors and SS samples from SCD. Hemoglobin profiles of samples were verified using the reference standard HPLC (VARIANT II, Bio-Rad Laboratories, Inc Hercules, California). The results were given as a percentage of HbS, A2, F, and the remainder was considered HbA since the values were normalized to 100%. Intracellular hemoglobin concentration for all the samples was measured under nomoi with CBC (Hema vet 950FS, Hematology System, Draw Scientific Inc; Miami, Florida).

### Sample Preparation

Three independent sample preparations were performed to test the different hypotheses. 1) whole blood, 2) isolated red blood cells (RBC) and 3) purified hemoglobin (lysed samples).

### Whole Blood Sample Preparation

75 μl of whole blood was mixed with 22 ml of pH 6.86 buffers and incubated for 10 minutes at room temperature before deoxygenation. Each microplate well used for analysis contained 12 μl of cells suspension that were diluted 300-fold from whole blood diluted in a buffer solution.

To create Vox+ samples 75 μl of undiluted blood was combined with 67 mg/mL voxelotor (Selleck chem) in 100% dimethyl sulfoxide (DMSO) (Sigma-Aldrich) to a final concentration of 30 μmol/L voxelotor that was determined based on pervious publication. The Vox+ samples used in this investigation were incubated for 1 hour with voxelotor at 37° C. temperature.

### RBC Sample Preparation

Whole blood was centrifuged at 500 g for 10 minutes in a micro centrifuge (model 21r; Thermo Scientific, Waltham, MA). The plasma and buffy coat were removed via aspiration. The RBCs were then washed three times with phosphate buffered saline (PBS) at pH 7.4. (Gibco, Thermo scientific, Waltham, MA).

45 μL of the washed RBC suspension was diluted with a pH 6.86 buffer solution, until the baseline hematocrit of 0.2% was achieved. Prior to deoxygenation, RBCs were incubated for 10 minutes in a pH 6.86 buffer solution at room temperature.

### Purified Hemoglobin (Lysed Samples) Preparation

Lysed samples were prepared as follows. Hemolysis was carried out by sonication for 30 second (Hemet Health; Portland, OR). Following hemolysis, the lysed cell suspension was added to various pH buffers solutions and centrifuged at 2000 g for 1 hour. Three hundred μl of the cell lysate was removed and the supernatant was discarded. Cell lysates were further diluted in 700 μl of the specific pH buffer solution and centrifuged at 2000 g for 1 hour. The 1 ml mixture of pH buffer solution and cell lysate were then filtered through a 0.02 um Millipore membrane.

The hemoglobin concentration in the lysed sample was determined using the hemoglobin cyanide (HiCN) method and measured with a cuvette spectrophotometer at 540 nm. Each of the purified lysed samples was diluted with different pH solutions (6.86, 7.2, 7.4, 8.0, and 10.0) to achieve a hemoglobin concentration of 4.36 μM, which had been determined as the baseline for lysed sample analysis.

### pH Measurements for the Buffer and Samples

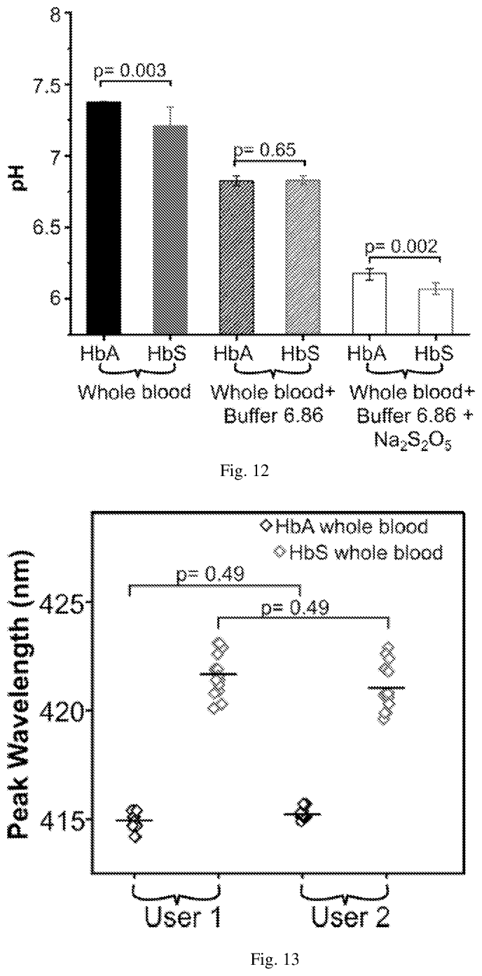

The pH of the buffer solutions was checked before and after additional to the prepared samples with an acumen AE150 pH meter (Fisher Scientific Waltham, MA). Using the same pH meter, we also measured the pH of a mixture of buffer, whole blood samples before and after additional of Na₂S₂O₅ (FIG. 12).

### Oxygenation and Deoxygenation of the Samples

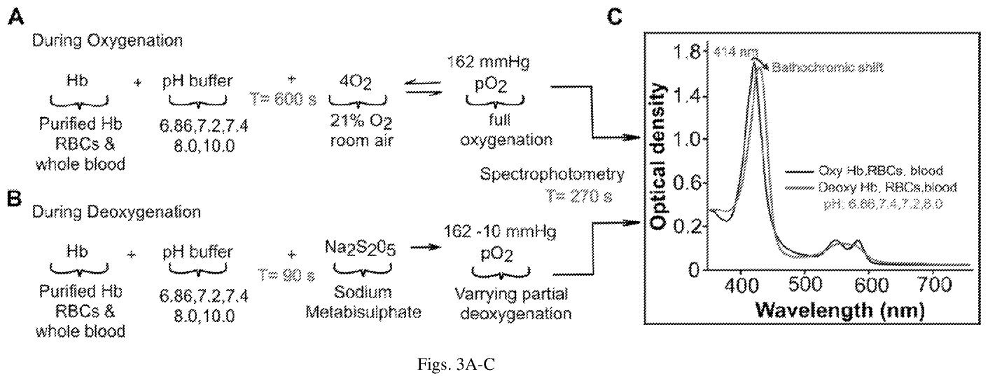

All samples were initially exposed to ambient air for 600 seconds. The oxygen partial pressure (PO₂) which is 21% of atmospheric pressure (773 mmHg), was then normalized to 162 mmHg PO₂ during oxygenation. (FIG. 3A). Sodium metabisulphite (Na₂S₂O₅) was used to deoxygenate the samples chemically. Predetermined amounts of sodium metabisulphite were mixed with 2 ml of the prepared samples for 90 seconds using a vortex (Scientific Industries, Bohemia, NY). The concentrations of Na₂S₂O₅ used in the testing were 0.039 M, 0.046 M, 0.053 M, 0.059 M, 0.066 M, 0.079 M, and 0.092 M, which corresponded to a gradual reduction in the oxygen partial pressure in the samples (FIG. 3C). A blood gas analyzer (Nova Starter Pro, Nova BioMed, Boston, Massachusetts) was used to confirm the deoxygenation levels at those Na₂S₂O₅ concentrations.

### Absorption Peak Wavelength Shift Assay

During sample testing of either oxygenated and deoxygenated samples, samples were analyzed in three microplate wells using a spectroscopy microplate reader Petromax Me2, (Molecular devices, San Jose, CA), over a spectral range of 350-750 nm, with a wavelength resolution of 2 nm and at a customized microplate well reading setting and at room temperature. Each acquisition lasted 270 seconds and the microplate was shaken for 5 seconds by the plate reader before each read.

### Sequence of the Assay:

1) Initially, the fully oxygenated samples were analyzed, to obtain reference signatures that included a Soret band (380-480 nm) and two peaks in Q-band (560-580 nm). All oxygenated samples had their highest absorption oxygenated peak at 414 nm (FIG. 1B).
2) Following that, the deoxygenated samples were analyzed across the same spectral range as the oxygenated samples, but with Na₂S₂O₅ concentrations ranging from 0.039 to 0.092 M. The concentration of Na₂S₂O₅ used was corresponding to a decrease from 100-0 (mmHg) of pO₂ in samples. The spectral of deoxygenated samples yielded two distinct peaks at pO₂ reduction of <90 mmHg in the sample. The largest peak found at 414 nm in the oxygenated spectral range was shifted, and the bathochromic shift varied depending on the type of sample under analysis and the pH buffer added with the sample, whilst the two peaks in the Q band range converged into a single peak at 560 nm (FIG. 3C). At pO₂ reduction >90 mmHg in the sample, the spectral yielded three different peaks; the bathochromic shift from 414 nm and the two peaks at 540 nm and 570 nm with a reduced gap between but the hump still exists.
3) For each sample and at each concentration of Na₂S₂O₅, the bathochromic wavelength shift and intensity with deoxygenation were assessed for further analysis

### Rate of Deoxygenation Assay

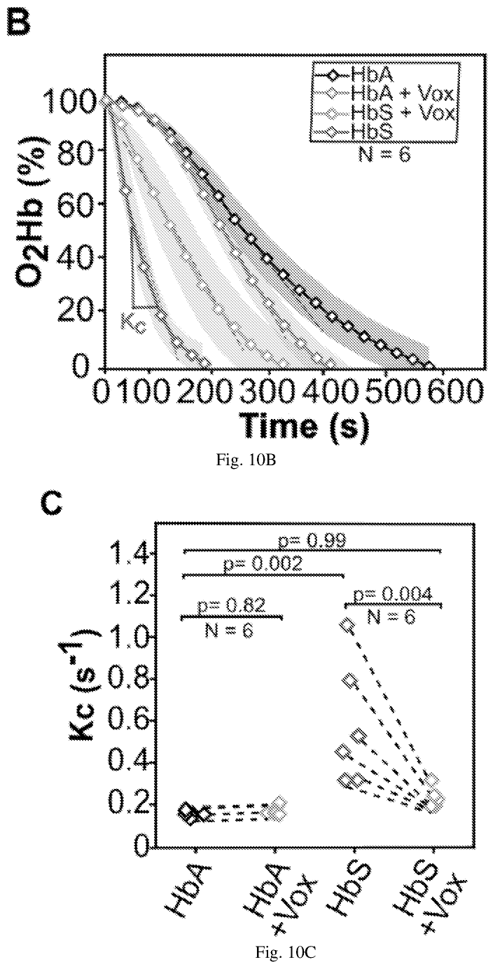

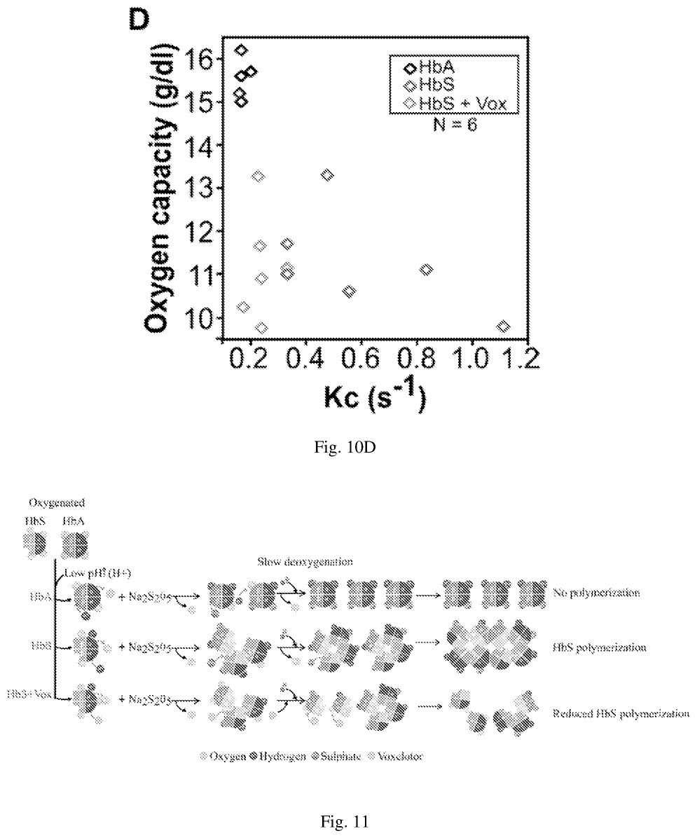

The hypochromic and bathochromic shift were obtained every 30 seconds for 600 seconds and used to determine the time course of deoxygenation of whole blood sample (FIG. 10A). Prepared whole blood samples were analyzed in three microplates wells using a spectroscopy microplate reader Spectromax Me₂, (Molecular devices, San Jose, CA) set at room temperature. As the whole blood, buffer and Na₂S₂O₅ equilibrated during deoxygenation, the corresponding bathochromic shift and hypochromic shift were recorded at interval of every 30 seconds (FIG. 10A). The rate of deoxygenation vs the time data was analyzed by determining the rate of constant Kc (FIG. 10B). The Kc was determined according to Dalziel and Lawson, but with a modification in the calculation. Kc as defined in previous studies is the fraction of total deoxygenation that occurs per second and calculated from the slope of line tangent to the time course of deoxygenation plot as defined by:

$$\frac{- d ( {O}_{2} Hb )}{dt} = {K}_{C} ( {O}_{2} Hb )$$

where t, is in seconds, and O₂Hb the concentration of oxygenated hemoglobin in whole blood sample

### Data Analysis

### Oxygenated and Deoxygenated Absorption Spectra

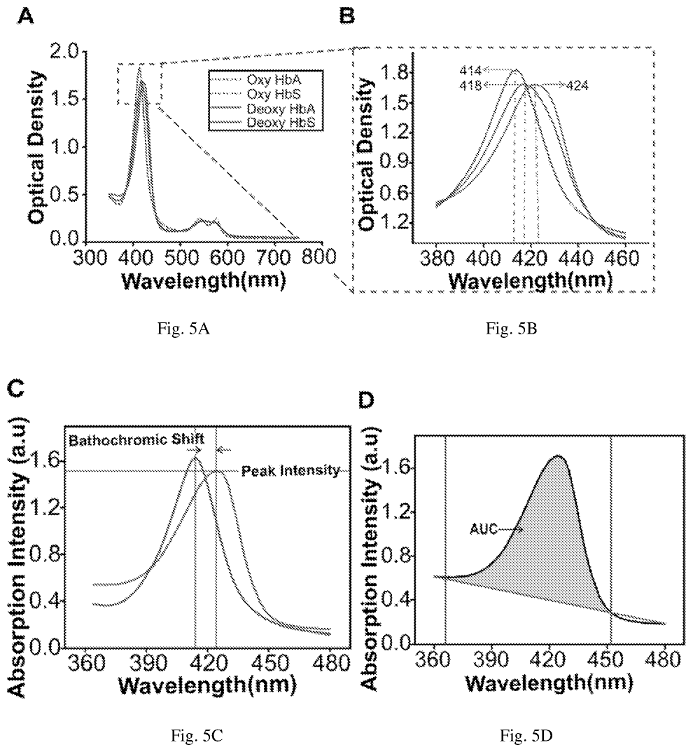

Oxygenated and deoxygenated spectra were obtained and processed in Softmax Pro 6.3. Individual peak, intensity and the peak wavelength shifts were identified from the spectra through an automated peak and intensity search (FIGS. 5A & C). Using SpectraGryph 1.2, the Soret peak was then baselined, smoothed, and examined. The area under the peak (380-460 nm) and full width at half maximum (FWHM) were then calculated from the normalized Soret peak (FIGS. 5D & E).

### Statistical Analysis

We used minitab software (Release 2021, Version 20; Minitab) for the statistical analysis. Mann Whitney U-test was performed to compare spectral variables between the normal and sickle groups. Pearson's correlation analysis was used to assess correlation between spectral variables, P<0.05 was considered statistically significant. Levene's test was performed to determine the homogeneity of variance between samples treated with voxelotor and those not treated. A paired t-test was performed to compare paired groups before and after voxelotor treatment and at the different temperature analyzed. Unless otherwise noted, results were expressed as means±standard error of the mean (SEM). Full set of 201 intensities from 350-750 nm (UV-Visible range) at 20% pO₂ and the four optical variables (peak wavelength shift, intensity, FWHM and area under the peak) data from normal and sickle blood sample were pooled and subjected to two-dimensional principal components analysis (PCA) using R Studio (Release 2021; 4.1.1) independently. The first and second principal components were PC1 and PC2, respectively. The two components were then clustered and visualized to represent the sickle and normal samples, respectively.

### Results

Sodium Metabisulphite (Na₂S₂O₅) Induces Whole Blood Oxygen Partial Pressure (pO₂) Decrease in Dose and pH Dependent Manner

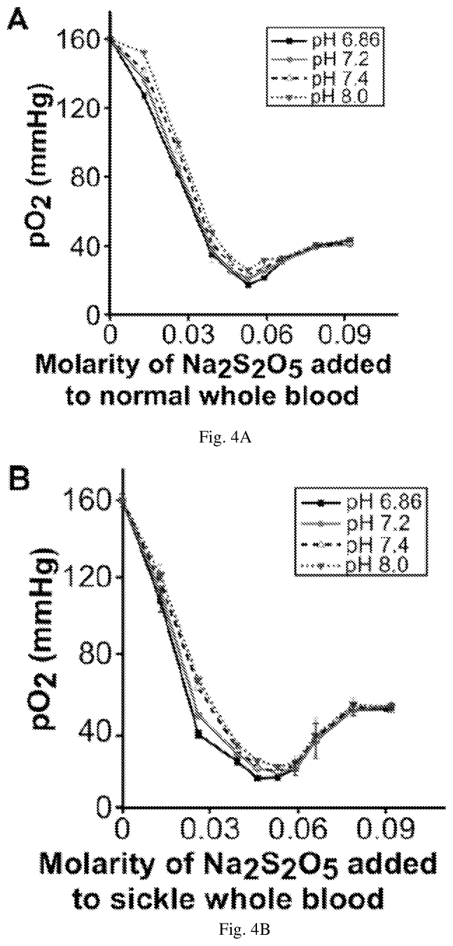

We used Sodium metabisulphite (Na₂S₂O₅) to deoxygenate hemoglobin and blood. To quantify the effect of Na₂S₂O₅ on deoxygenation (FIG. 3), we measured the oxygen partial pressure (pO₂) in both normal and sickle cell whole blood samples reconstituted with a range of Na₂S₂O₅ concentrations from 0 to 0.092 M at physiologic and supra-physiologic pH (buffer pH values: 6.86, 7.2, 7.4, 8.0, and 10.0). At all pH levels, the pO₂ levels of both normal and sickle whole blood samples were determined to be at 162 mmHg prior to addition of Na₂S₂O₅ (FIG. 4). The pO₂ levels of both normal and sickle whole blood samples decreased with increasing Na₂S₂O₅ concentration from 0.039 M to 0.053 M, followed by a moderate increase from 0.053 M to 0.066 M, and reached quasi-equilibrium state from 0.079 M and above (FIG. 4A, B).

Under all Na₂S₂O₅ concentrations, the pO₂ of both normal and sickle whole blood decreased with decrease in pH (FIG. 4A, B). Sickle whole blood demonstrated lower pO₂ levels compared to normal whole blood under the same Na₂S₂O₅ concentration and pH value, except at zero Na₂S₂O₅ concentration (max. PO₂=162 mmHg) and 0.053 M (min. PO₂ around 10 mmHg) (FIG. 4B). Together, these results demonstrate that 1). Increase in the concentration Na₂S₂O₅ and decrease in pH decreases pO₂ levels in blood samples and 2) pO₂ levels in sickle blood samples are subjected to a higher decrease than normal blood samples when treated with Na₂S₂O₅

Purified Sickle Hemoglobin's Bathochromic Peak Wavelength Shifts More than Normal Hemoglobin Upon Deoxygenation

Both oxygenated HbS and HbA purified hemoglobin exhibited 3 distinct peaks at 414 nm and two-valley peaks at 540 and 576 nm (FIG. 5A). At pO₂=28 mmHg, the peaks at 540 and 576 nm converged at approximately 560 nm to form one broad peak. The maximum 414 nm peak observed in oxy-HbS and oxy-HbA shifted to different extents upon deoxygenation such that the shift of HbS was greater (424 nm and 418 nm respectively (FIGS. 5B, C). It was noted that the trends of changes in the wavelength with deoxygenation was similar to the trend of deoxygenation with Na₂S₂O₅ (FIG. 4) indicating that bathochromic shift is a surrogate marker for deoxygenation. At 162 mmHg pO₂, both normal and sickle whole blood had a peak wavelength of 414 nm. The reduction in PO₂ from 162 to 10 mmHg in both normal and sickle whole blood caused an increase in shift and quasi-equilibrium was observed at 28 mmHg pO₂ (FIG. 5C). Using the peak wavelength shifted curve the peak intensity, area under the curve and FWHM were computed (FIGS. 5D, E). These findings demonstrated that using the deoxygenated curve four district optical parameters could be formulated.

### Purified Hemoglobin Demonstrates Increased Optical Absorption Bathochromic Shift Upon Deoxygenation Through a Wide Range of pH Values

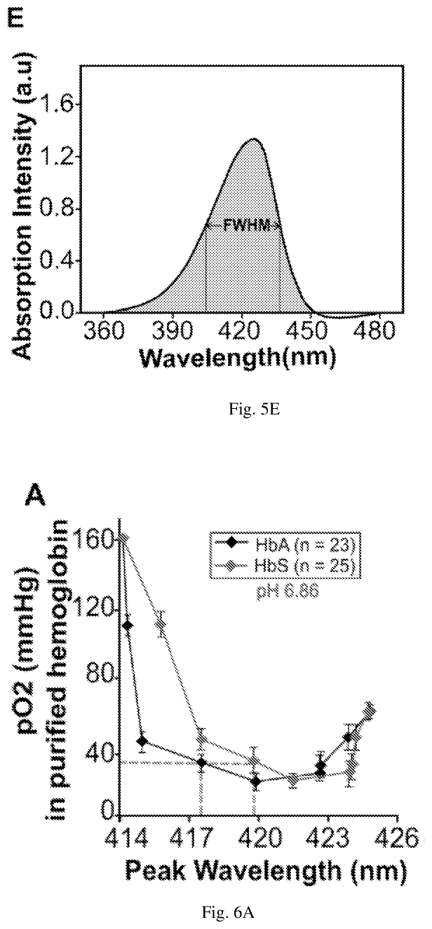

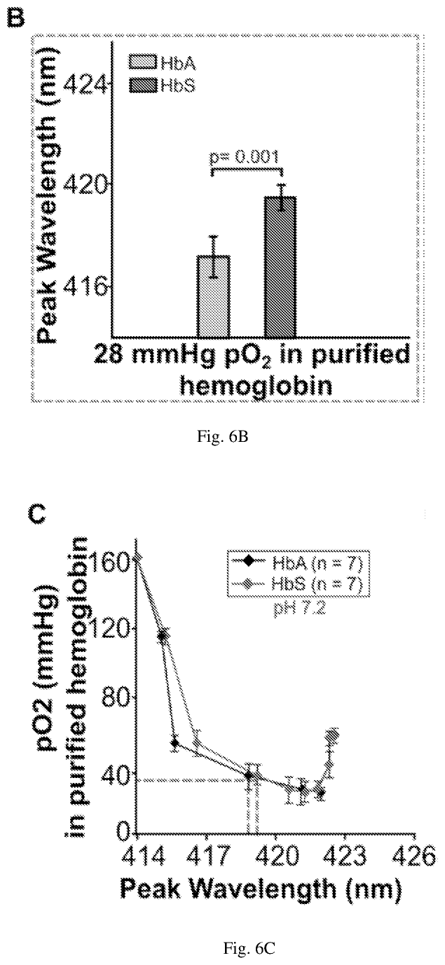

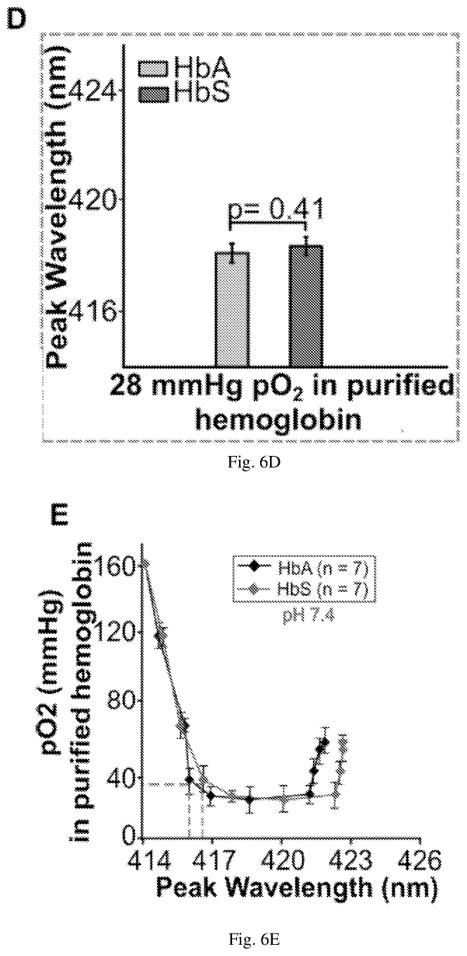

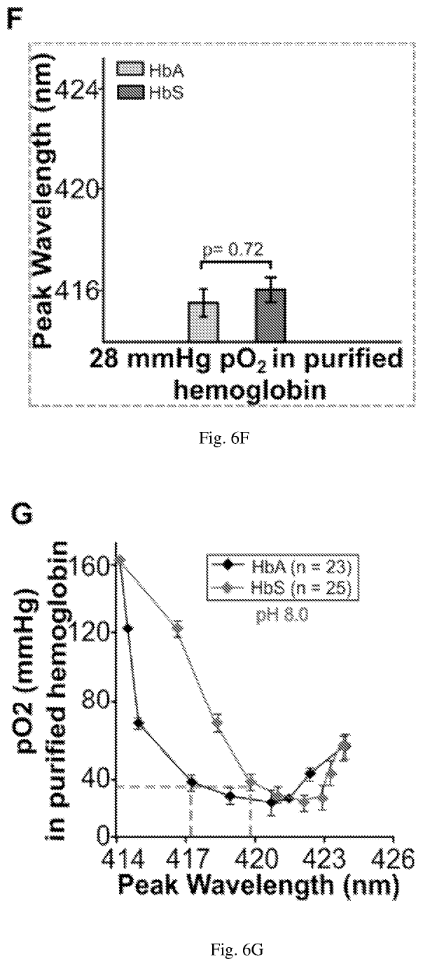

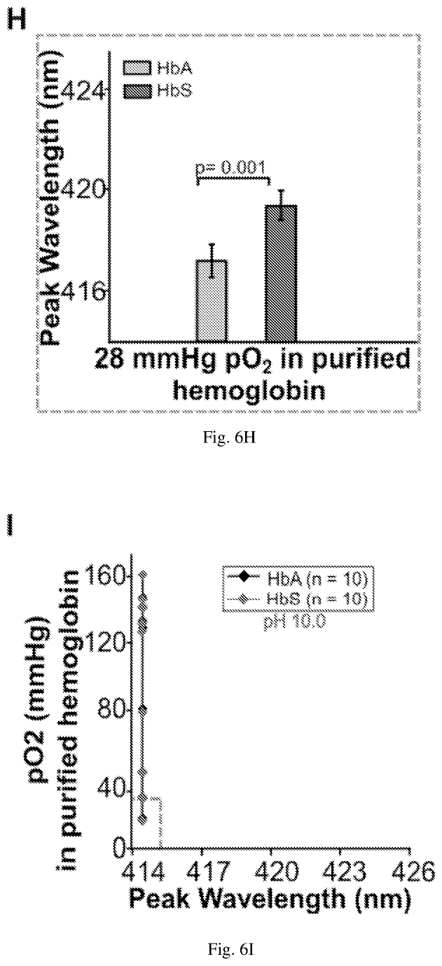

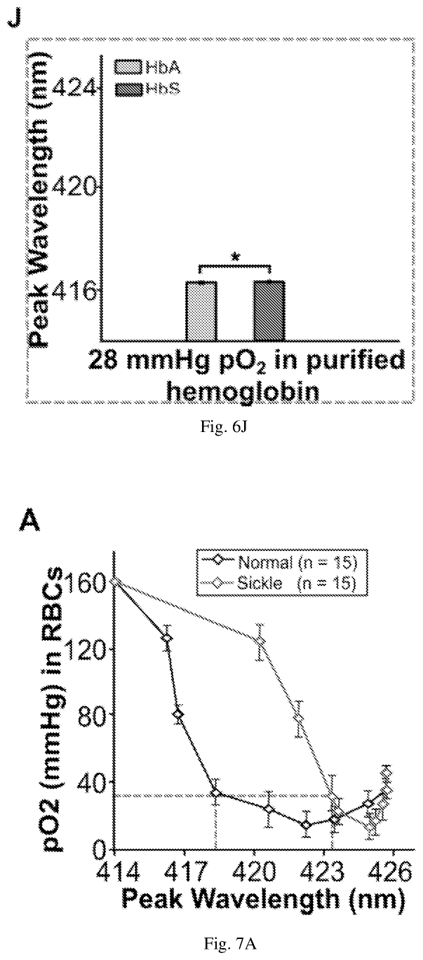

At all pH values, fully oxygenated HbA and HbS had the same peak wavelengths of 414 nm (FIG. 6). At all pH values except pH=10, decreasing the pO₂ from 162 to 20 mmHg induced peak wavelength shifts in both HbA and HbS, followed by a plateau from pO₂ 20 to 10 mmHg. At pO₂, 28 mmHg, substantial variations in peak wavelength shifts between HbA and HbS were found at pH=6.86 (FIG. 6B) (p=0.001) and pH=8 (FIG. 6G) (p=0.001). At pH=7.2 (FIG. 6D) (p=0.41) and pH=7.4 (FIG. 6F) (p=0.72), there were no significant changes in peak wavelength shifts between HbA and HbS. At pH=10 (FIG. 4J), no bathochromic shifts were found regardless of the increase in Na₂S₂O concentration, and any differences were not significant. These results demonstrated that pH had an influence on the magnitude of bathochromic shift for both HbA and HbS purified hemoglobin. For both HbA and HbS purified hemoglobin, highest magnitude of bathochromic shift was obtained at pH=6.86, followed by at pH=8.0 and the least magnitude of bathochromic shift was obtained at pH=10. At all pH values except pH=10, HbS had a highest magnitude of bathochromic shift compared to HbA. In conclusion, bathochromic shifts at pH=6.86 and pH=8 could be used to confidently differentiate between HbA and HbS.

RBCs and Whole Blood Samples from Patients with SCD Demonstrate Increased Optical Absorption Bathochromic Shift Compared to Samples from Normal Donors

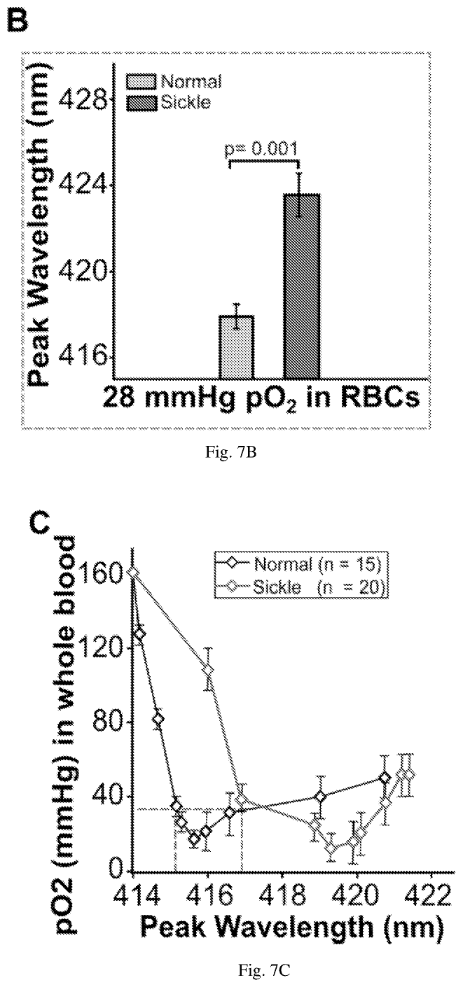

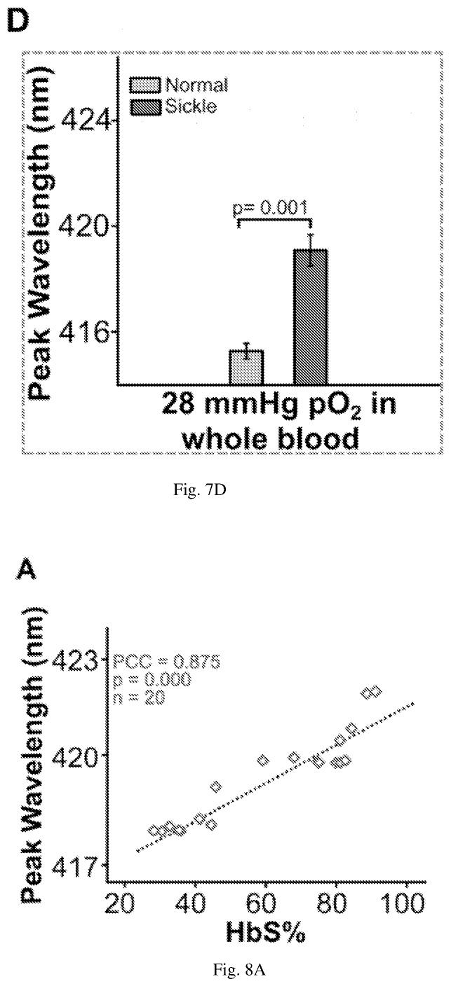

For the analysis of RBCs and whole blood samples, we focused our analyses on pH 6.86 because the highest magnitude of peak wavelength difference between HbA and HbS purified hemoglobin was obtained at this pH value. The variations of bathochromic shift shown for purified hemoglobin was also observable for RBCs (FIG. 7A) and whole blood (FIG. 7C). RBCs and whole blood from normal and SCD patients also exhibited the three wavelengths that were present in purified hemoglobin. From 162 to 10 mmHg pO₂, decrease in pO₂ induced a rise in both normal and sickle samples peak wavelength for both RBCs and whole blood, followed by a plateau from 20−10 mmHg. At 28 mmHg pO₂, bathochromic shifts between normal and sickle RBCs (FIG. 7B) (p=0.001) and normal and sickle whole blood (FIG. 7D) (p=0.001) were significantly different. Compared to bathochromic shift of purified hemoglobin for both HbA and HbS at pH 6.86, RBCs had the largest magnitude of bathochromic shift, followed by purified hemoglobin and then whole blood samples. Bathochromic shift summaries are shown in Table 1. Sickle RBCs and whole blood had a larger bathochromic shift compared to normal RBCs and whole blood. The findings demonstrate that the bathochromic shift difference shown by purified hemoglobin HbA and HbS is also observable when using analytes like RBCs and whole blood.

TABLE 1
Optical peak wavelength of the different analyte
at pH 6.86 and pO₂ = 28 mmHg
Peak wavelength

| | Normal sample | Sickle sample |
|---|---|---|
| Purified hemoglobin | 417.2 ± 0.78 | 420.3 ± 0.52 |
| RBCs | 418.0 ± 0.56 | 422.9 ± 0.63 |
| Whole blood samples | 415.3 ± 0.23 | 419.6 ± 0.48 |

*Data is mean ± SEM.

Characteristics of Whole Blood Optical Absorption Measure Demonstrated Association with Hemoglobin Levels and Percentages of Sickle Hemoglobin

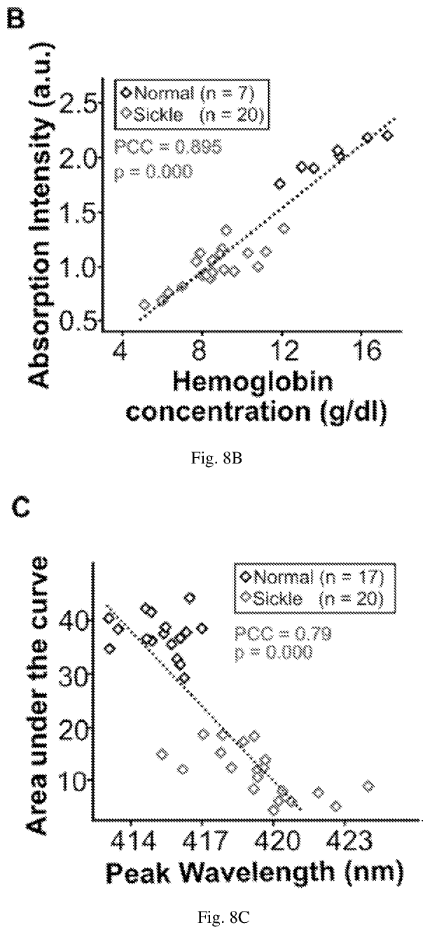

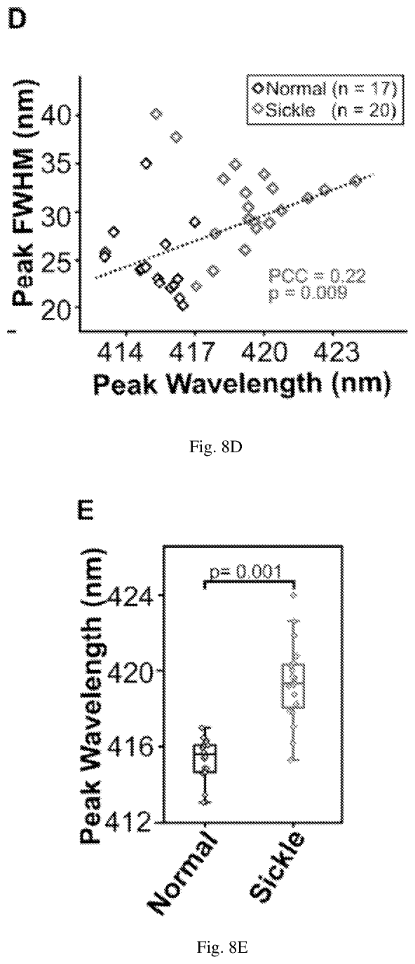

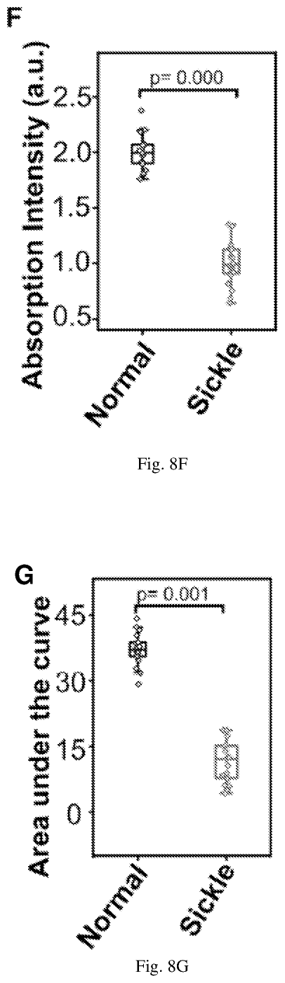

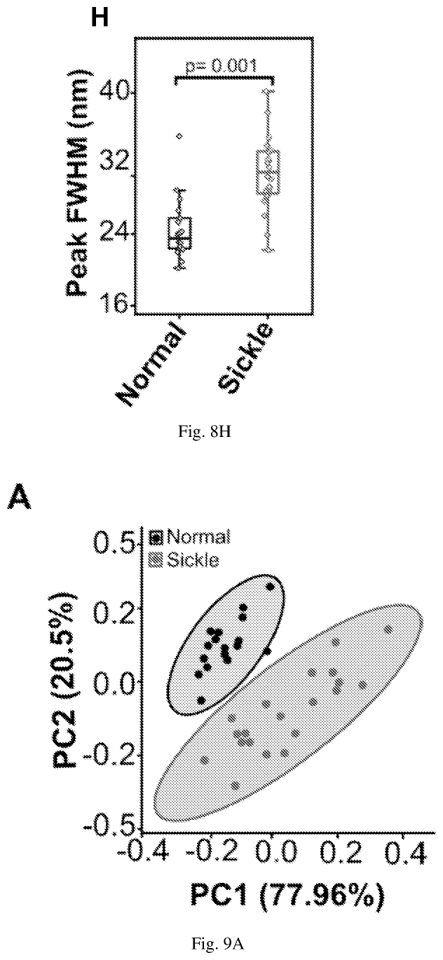

Aside from obtaining the measurable optical variables and examining their confounding effects, it was also critical to fully comprehend the association between absorption bathochromic shift and intensity, with HbS fraction and hemoglobin concentration. Sickle whole blood bathochromic shifts were highly associated with the changes in percentage of hemoglobin S (HbS %) obtained using High Performance Liquid Chromatography (HPLC) for each whole blood sample at pH 6.86 and pO₂ 20% (FIG. 8A) (PCC=0.875, p=0.000, n=20). The absorption intensity for whole blood samples at pH 6.86 and 28 mmHg mpO₂ (FIG. 8B) was strongly and positively associated with the concentration of hemoglobin (g/dl) obtained by the standard Complete Blood Count (CBC) for both normal and sickle whole blood samples (PCC=0.89, p=0.000, sickle (n=20), normal (n=7). At 20% pO₂ (FIG. 8C), the area under the shifted peak was significantly and inversely associated with peak wavelength shift in both normal and sickle whole blood (PCC=−0.79, p=0.000, sickle (n=20), normal (n=17). (FIG. 8D) FWHM were significantly but not strongly linked with peak wavelength shift for both normal and sickle whole blood, (PCC=0.22, p=0.009, sickle (n=20), normal (n=17)). Additional correlations between the variables and physiological importance are shown in Table 2. By comparing the optical variables of normal and sickle whole blood independently, we noted that sickle whole blood had a higher magnitude of bathochromic shift and FWHM compared to normal whole blood: (sickle; 420.6±0.44, normal; 415.3.0±0.21), (sickle; 30.9±0.84, normal; 24.3±0.82) respectively, normal whole blood had a higher shifted peak optical intensity and area under the shifted peak compared to sickle whole blood (sickle:0.99±0.04, normal; 2.0±0.03), (sickle: 11.6±0.69, normal; 37.2±0.8) respectively. There was a significant difference between normal and sickle whole blood for all optical variables. (FIG. 68E) peak wavelength shift (p=0.001), (FIG. 8F) peak intensity (p=0.000), (FIG. 8H) area under the shifted peak (p=0.001), and (FIG. 8G) shifted peak FWHM (p=0.001). These results revealed that magnitude of bathochromic shift could be used to predict the percentage of HbS in sickle samples, absorption intensity for determination of the concentration of hemoglobin, area under the curve to determine anemia status and FWHM to assess the homogeneity of the sample. When compared to normal samples, the homogeneity of sickle whole blood was less consistent, since sickle patients were on different therapies which lead to varying fraction of HbS.

TABLE 2
Correlations between the optical variable and physiological significance

| | | | | | Physiological | |
|---|---|---|---|---|---|---|
| | Peak | Peak | Area under | peak | Parameter | Physiological |
| | wavelength | intensity | the curve | FWHM | Correlated | significance |
| Peak | 1 | | | | Percentage | Identification |
| wavelength | | | | | of variant | of Variant SS |
| | | | | | hemoglobin SS | |
| Peak | PCC = −0.46 | 1 | | | Blood | Determine |
| intensity | p = 0.000 | | | | concentration | the level of |
| | Negative | | | | of Hemoglobin | Hemoglobin |
| | | | | | (g/dl) | level |
| Area under | PCC = −0.76 | PCC = 0.94 | 1 | | Anemia (low | Identification |
| the curve | p = 0.000 | p = 0.000 | | | hemoglobin | of Anemia |
| | Negative | Positive | | | detection) | |
| Peak | PCC = −0.22 | PCC = −0.41 | PCC = −40.0 | 1 | Sample | Variation in |
| FWHM | p = 0.006 | p = 0.000 | p = 0.000 | | homogeneity | samples |
| | Negative | Negative | Negative | | | |

*PCC: Pearson Correlation Coefficient, p-values were obtained from linear regression model.

Principal Component Analysis (PCA) Differentiates Normal from Sickle Whole Blood Samples Based on Measured Optical Absorption Variables and Deoxygenated Spectra Intensity

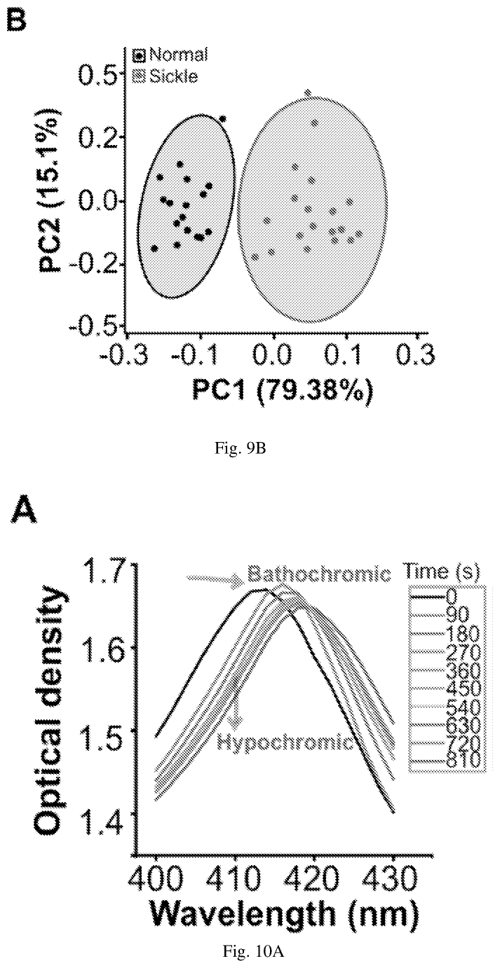

The two principal components obtained accounted for 98.5% variability in the original full spectrum peak intensity data set, with the first component accounting for 77.96% of the total variation (FIG. 9A). The two components account for 94.5% variability in the optical parameters data set, with the first component accounting for 79.38% of the total variation between normal and sickle whole blood samples (FIG. 9B). Only one outlier was detected in the full spectrum data set in normal whole blood samples, whereas two were found in the four optical parameters dataset in normal and sickle whole blood samples, respectively. The PCA dimension reduction and clustering clearly showed grouping in normal and sickle intensity analysis and when using the four variables which diverged from the shifted wavelength.

### Voxelotor Induced Reduction in Kc and Heterogeneity in Whole Blood Samples Containing Sickle Hemoglobin at pH 6.86

The magnitude of hypochromic shift was found to correlate to the magnitude of bathochromic shift between 400 and 430 nm wavelength. As the magnitude of bathochromic shift increased, the magnitude of hypochromic shift increased. The time course of deoxygenation or rate of deoxygenation was obtained from assessing the hypochromic and bathochromic shift every 30 seconds interval for 600 seconds (FIG. 10A). The first order rate of deoxygenation plot of (O₂Hb) against time for sickle and normal whole blood samples were linear to concentration of oxygenated hemoglobin (O₂Hb) from 100% to 0% oxygen saturation (FIG. 10B). The fraction of total deoxygenation that occurs per second Kc (see⁻¹) was calculated as previously reported from the slope of the line tangent to the plot of time course of deoxygenation (FIG. 10B). We observed that whole blood samples with HbS had increases values of Kc compared to HbA whole blood samples and the difference was significant with p=0.002 (FIG. 10C). However once HbS samples were incubated with voxelotor at a concentration of 30 μmol/L, the Kc was decreased by 57.5%±6.7%. Kc value for HbS and those of HbS treated with Vox (Hb S+ Vox), were significant different (p=0.004). On the other hand, HbA samples incubated with voxelotor (HbA+ Vox), there was no significant different from HbA (p=0.82). Kc was also found to have an association with the oxygen capacity (g/dl) (FIG. 10D). HbA containing samples had a higher oxygen capacity but with low Kc values while HbS containing samples had lower oxygen capacity but with high Kc values that have a higher heterogeneity. However, HbS+ Vox, had a reduced Kc value but with a low oxygen capacity as HbS without voxelotor. HbS+ Vox had a reduced heterogeneity that was statistically significant compared to HbS (P=0.006; Levane homogeneity of variance test). Those results indicate that whole blood samples containing HbS have an increased rate of deoxygenation compared to whole blood samples containing HbA. However, when HbS samples were incubated with voxelotor, voxelotor reduced the rate of deoxygenation by increasing the oxygen affinity and thus increasing the time of deoxygenation. Voxelotor reduced the Kc value and improved the heterogeneity of HbS sample but does not improve the oxygen capacity.

### Rigor and Reproducibility

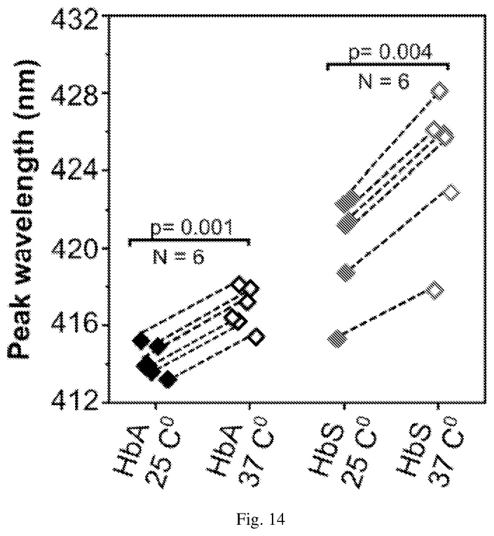

The repeatability of bathochromic shift was established by comparing variations between two users in 20 experiments using the same whole blood samples at approximately 20 mmHg pO₂ (FIG. 14). There were two samples used in this study (one normal and one sickle). Each sample was evaluated five times, and the same sample was analyzed in three-microwells throughout each test, resulting in a total of ten tests for each user. The peak wavelength shifts between the two users showed excellent repeatability ((Mean±SEM), (User1: sickle 421.6±0.24, normal; 414.9±0.11; User2: sickle 421.1±0.26, normal; 415.1±0.05) and coefficient of variance (COV)=0.05% for sickle and 0.02% for normal). bathochromic shifts between User 1 and User 2 for both normal and sickle whole blood (p=0.07) and (p=0.68) were not significantly different. These results indicate reproducibility and a good precision of the assay.

We describe a rapid optical method that can detect HbS, anemia and measures Hb oxygen affinity, rate of deoxygenation and Hb concentration for whole blood samples, determined at a controlled deoxygenation levels and pH. We found that the optical absorption spectral of HbA and HbS differed under certain deoxygenation and pH circumstances, based on actual spectral bathochromic shifts and hypochromic shifts. The bathochromic shifts of HbA and HbS purified hemoglobin's were significantly different at pH 6.86, and no bathochromic shift appeared at pH 10.0, revealing that under more alkaline conditions above pH 8.5, the Bohr effect does not hold true, for HbS and the oxygen affinity of HbS is similar to that HbA irrespective of the hypoxia levels. These findings provide a clear explanation and mechanism behind the attempts of previous investigators to treat sickle cell disease crises by alkalizing the blood.

We show that the oxygen affinity of Hb in whole blood can be measured using optical bathochromic shifts. SCD samples had a larger bathochromic and hypochromic shift magnitude under deoxygenation at all pH levels compared to normal healthy blood containing HbA, where the higher the magnitude of the shift, the lower the oxygen affinity of HbS. The magnitude of bathochromic shift in SCD samples significantly correlated to the percentage of HbS. This explains that the lowered oxygen affinity of HbS is caused by polymerization (HbS concentration dependent process) and decreased pH. Low pH causes an increase in hydrogen ions binding to histidine amino acids such as β-146 and α-122, displacing oxygen and promoting the formation of salt bridges that stabilize hemoglobin in the deoxygenated state (FIG. 11). Once HbS is deoxygenated, it becomes adhesive and forms polymers (FIG. 11), reducing its oxygen affinity even further.

We also have found that shifted peak intensity is positively correlated with the hemoglobin concentration and the bathochromic shift had a negative correlation with the area under the peak. However, no correlations are related with FHWM, but the results were statistically significant. These results revealed that other than the detection of HbS and assessment of oxygen affinity, this method can be used to optically determine the hemoglobin concentration and homogeneity of the sample as well as detection of anemia, a common condition in patients with SCD.

Although we have focused mainly on assessment of oxygen affinity, the developed method can also be used to study the kinetics of whole blood deoxygenation. We demonstrated the effect of an oxygen modifying drug, voxelotor that has recently been approved for treatment of SCD. We found a significant difference in the rate of deoxygenation with SCD whole blood samples treated with voxelotor and samples that were not treated. Although voxelotor could reduce the rate of deoxygenation for SCD samples, deoxygenation rate was still lower than for normal healthy patients (FIG. 10). It was also found that voxelotor reduces the rate of deoxygenation heterogeneity of SCD. However, voxelotor had a reverse impact on HbA at relatively low hemoglobin oxygen concentration which indicate that voxelotor negatively impact the oxygen affinity normal healthy RBC. Although voxelotor increased the rate of deoxygenation in SCD samples, the oxygen capacity of SCD blood was not increased. These results accord with the investigators that report that although voxelotor increases the oxygen affinity, it may not increase the oxygen delivery. Since the effect of voxelotor is still unclear. Further studies will be needed to clearly understand the effect of voxelotor on HbA and its effect on oxygen delivery of HbS.

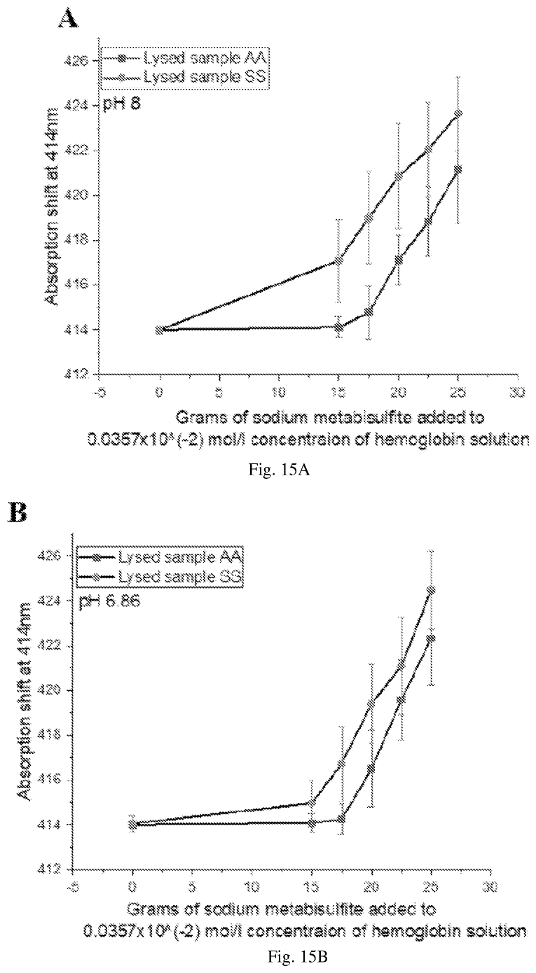

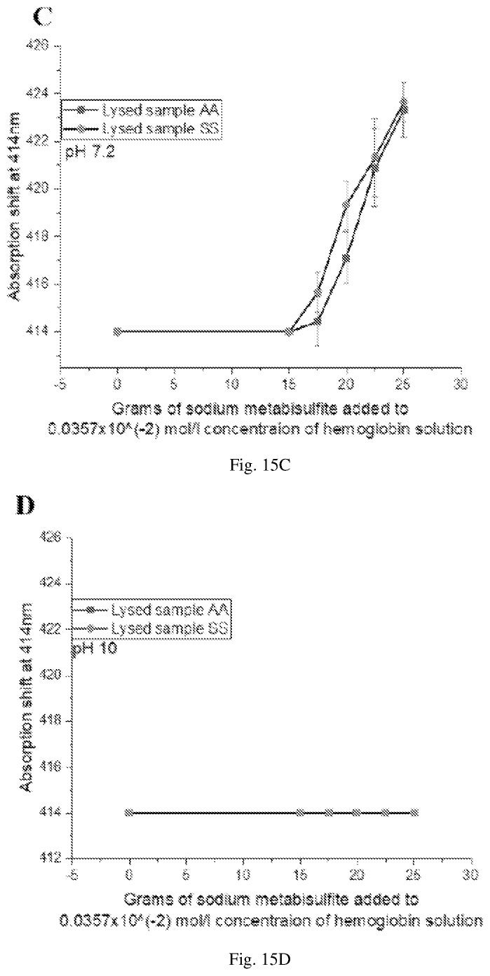

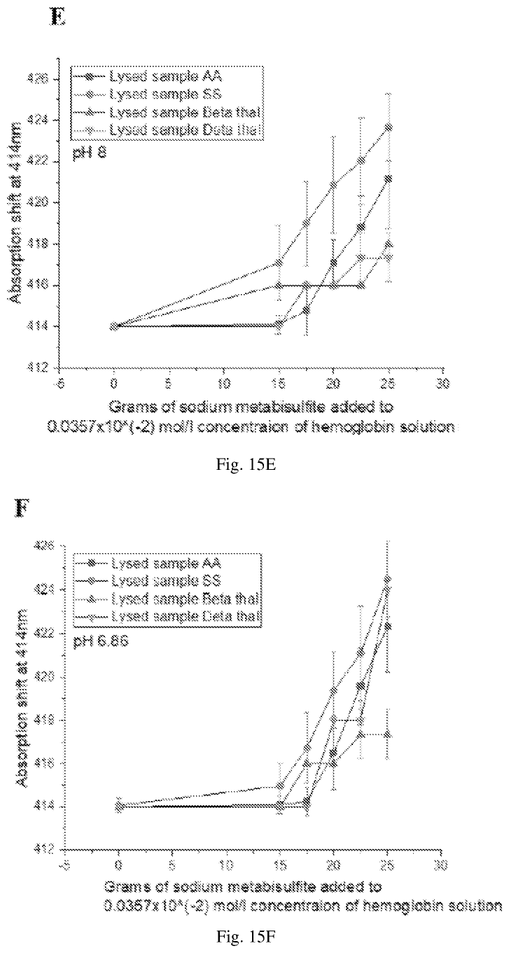

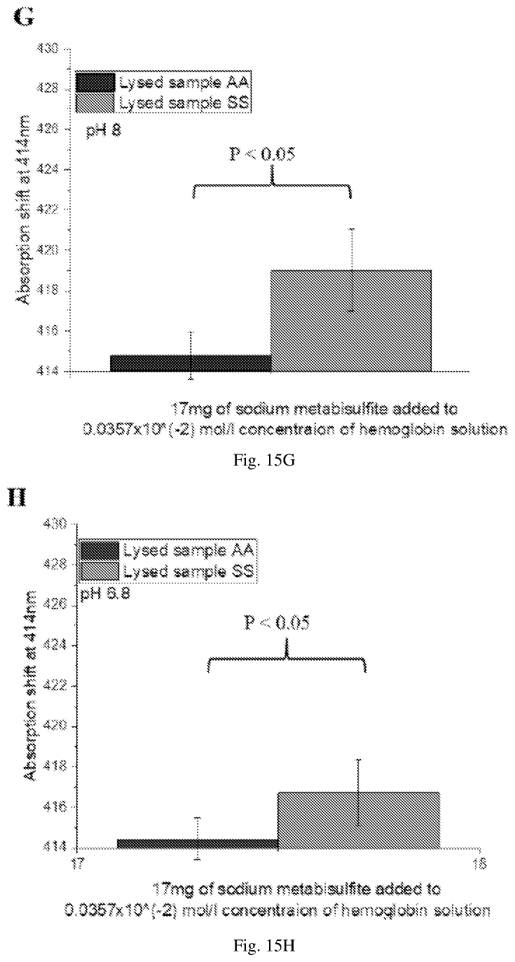

While methods of detection of HbA and measurement of the oxygen affinity and rate of deoxygenation were conducted at room temperature, this method was also tested at an increased temperature of 37° C. We found that the magnitude of bathochromic shift was elevated at 37 C for both normal and sickle samples compared to room temperature (FIG. 15). However, the magnitude of bathochromic shift was more elevated in sickle samples at the same pO₂, these results agree with prior studies that report temperature having an impact on HbS polymerization.

This example provides support for using optical absorption parameters to differentiate hemoglobin variants, and motivates investigation to understanding HbS deoxygenation response dynamics and single cell RBC assessment that is critical in the recent genetic approach treatment for SCD. We provide evidence that could be used to improve the diagnostic accuracy of systems that use the current absorption spectrophotometer and are affected by variant testing. We show the influence of pH (Bohr effect) using a ubiquitous absorption method and provide convincing evidence that once the pH becomes more acidic and in the initial alkaline ranges, absorption peak wavelength shifts were detected, and these shifts were associated with HbS profile in the sickle cell blood sample. We also demonstrated how we could test the effectiveness of treatment for sickle cell disease by assessing the alteration in the rate of deoxygenation. We show that the magnitude of bathochromic shift relates to the pH which was also was dependent on the concentration of HbS.

The finding of a correlation between bathochromic shift in sickle whole blood samples and hemoglobin S percentages suggests that bathochromic shift can be utilized to estimate the profile of hemoglobin S in a sample. Our Principal component analysis (PCA) algorithm for the entire intensity spectrum and the four variables clearly showed distinguishable clusters of normal and sickle whole blood samples. The PCA results revealed that, while we originally clustered sickle blood from normal samples in this study, the assay may also be utilized to identify other hemoglobin variants based on their altered oxygen affinity, as shown by the acquired optical variables. These could include common hemoglobin variants with altered oxygen affinity for which no point-of-care diagnostic technology exists.

The effect of pH and thus increases Bohr effect was shown with the optical absorption peak for both HbS and HbA shifting under different pH conditions. We revealed that when the pH is lower than 7.4, the magnitude of the bathochromic and hypochromic shift for both HbS and HbA is greatest, indicating rapid oxygen dissociation from hemoglobin. However, as pH is increased to 8.0 and then to 10.0 the magnitude of bathochromic shift decreases as compared to pH of 6.86. The bathochromic shift at pH 7.4 or higher must have been contributed by the increased levels of 2,3 DPG leading to changes in the Donna equilibrium and thus lowering the pH. On another hand no bathochromic or hypochromic shift were observed at pH of 10.0

Considering the effect of pH on subsequent hemoglobin structural changes has been studied in the Bohr effect using the p50. In comparison to HbA, any change in pH beyond the physiological range of 7.35 to 7.45 amplifies the HbS Bohr effect, resulting in increased deoxygenation.

### Example 2

We discovered that when hemoglobin or RBC from a blood sample is placed in a pH buffer solution of 6.86, or 8.0, hemoglobin molecules undergo a conformational change that is based on the type of hemoglobin variant. The alternated hemoglobin variant molecules in a concentration range (4.36 μM) or 2% hematocrit are then chemically deoxygenated by adding sodium metabisulfite in a range (15-25 mg) to deplete oxygen from hemoglobin molecule and there after exposed to an optical beam of light in the UV-Visible wavelength range. The absorbed light by the hemoglobin-buffer solution yields unique optical signatures of absorption shifts and a reduction in the optical density that are unique to every hemoglobin variant under investigation. The shift and intensity ratios could then be used to identify hemoglobin variant and their oxygen bind capability.

### Results

We have recently used a validated bench top absorption spectroscopy analyzer to perform this proof of concept on nearly 47 patient blood samples (23 normal AA,23 hemoglobin variant SS, 2 S beta thalassemia and 2 delta beta thalassemia) (FIG. 15). We used a standard HPLC device to confirm relative protein compositions. Our preliminary results from 47 clinical samples have shown that under pH 6.86 and 8, absorption wavelengths of SS and thalassemia rich variants shift significantly more than that of AA-rich variants provided that certain thresholds of deoxygenation are induced in these samples (FIGS. 15A, B)). Compared to pH 10, there was not significate absorption wavelength shift difference between AA and SS,rich variants, implying that AA, SS and thalassemia are comparable under pH 10. Although there was a slight difference in absorption wavelength change between AA and SS, thalassemia rich variants, it was not significant at under pH 7.2. (FIGS. 15C, D). These results emphasize the exciting possibility that hemoglobin variants can be distinguished using light absorption shifts under pH 6.8 and 8 environments. (FIGS. 15E, F). We tested the optical shift difference obtained from SS, rich variants, AA-rich under pH 6.86 and 8 environment using a one-way Mann Whitney test and found a detectable statistical difference between the groups. (FIGS. 15G, H).

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: A method of determining at least one of hemoglobin oxygen affinity, rate of hemoglobin deoxygenation, or the presence of hemoglobin variants in blood of a subject, the method comprising:
determining differences of absorption spectra of oxygenated and deoxygenated hemoglobin, red blood, and/or blood obtained from the subject; and
comparing the determined absorption spectra differences to a control value, wherein the absorption spectra differences are indicative of hemoglobin oxygen affinity, rate of hemoglobin deoxygenation, or the presence of hemoglobin variants in the blood of the subject.

2: The method of claim 1, wherein the absorption spectra of the oxygenated and chemically deoxygenated hemoglobin are measured at the same pH from about 6.5 to about 9.0.

3: The method of claim 1, wherein the differences of absorption spectra are determined by:
generating a first optical absorption spectrum of oxygenated hemoglobin, red blood, and/or blood obtained from the subject;
generating a second optical absorption spectrum of deoxygenated hemoglobin, red blood, and/or blood obtained from the subject;
comparing the first optical absorption spectrum with second optical absorption to determine differences of the absorption spectra.

4: The method of claim 3, wherein the differences of the absorption spectra include at least one of a bathochromic shift and/or hypochromic shift in peak wavelength from the first absorption spectrum to the second absorption spectrum.

5: The method of claim 4, wherein the magnitude of barochromic shift in peak wavelength is indicative of at least one hemoglobin oxygen affinity, rate of hemoglobin deoxygenation, or presence and/or percentage of hemoglobin variants in the hemoglobin, red blood cells, or blood of the subject.

6: The method of claim 5, wherein an increase in magnitude of biochromatic shifty and/or hypochromic shift in peak wavelength is indicative of decreased hemoglobin oxygen affinity, increased hemoglobin deoxidation, or the subject having sickle cell disease.

7: The method of claim 4, wherein the differences of area under a curve of and/or full width half maximum of peak wavelengths of the first absorption spectrum and the second absorption spectrum are indicative of anemia of the subject and homogeneity of hemoglobin in the subject.

8: The method of claim 1, wherein the hemoglobin, red blood cells, and/or blood is chemically deoxygenated by mixing the hemoglobin, red blood cells, and/or blood with an amount of chemical deoxygenant effective to deplete oxygen from the hemoglobin.

9: The method of claim 8, wherein the chemical deoxygenant comprises sodium metabisulfite.

10: The method of claim 1, wherein the hemoglobin variant is selected from HbSA, HbSS, HbSC, and HbA2.

11: The method of claim 10, wherein detection of HbSA hemoglobin variant diagnoses the subject as having a sickle cell trait.

12: The method of claim 10, wherein detection of HbSS hemoglobin variant diagnoses the subject as having a sickle cell disease.

13: The method of claim 10, wherein detection of HbSC hemoglobin variant diagnoses the subject as having a hemoglobin SC disease.

14: The method of claim 10, wherein detection of HbA2 hemoglobin variant diagnoses the subject as having thalassemia.

15: A method of detecting hemoglobin variants in blood of a subject, the method comprising:
determining an optical signature of hemoglobin, red blood cells, and/or blood obtained from the subject that has been chemically deoxygenated and that has a pH from about 6.5 to about 9.0; and
comparing the determined optical signature to a control optical signature wherein differences between the determined optical signature and the control optical signature is indicative of hemoglobin variants.

16: The method of claim 15, wherein the optical signature is determined using UV-VIS light spectroscopy.

17: The method of claim 15, wherein the determined optical signature includes an absorption spectra of the deoxygenated hemoglobin, red blood cells, and/or blood.

18: The method of claim 15, wherein control optical signature includes an absorption spectra of chemically deoxygenated normal hemoglobin, red blood cells, and/or blood obtained at substantially the same pH as the optical signature of the hemoglobin, red blood cells, and/or blood obtained from the subject.

19: The method of claim 15, further comprising adding hemoglobin, red blood cells, and/or blood obtained from a subject to a pH buffer solution prior to determining the optical signature, wherein the hemoglobin, red blood cells, and/or blood added to the pH buffer solution undergoes a conformational change.

20: The method of claim 19, wherein the buffer solution has a weak acidic or weak basic pH.

21-34. (canceled)
